(19) United States
(12) Patent Application Publication
(10) Pub. No.: US 2002/0177180 A1
(43) Pub. Date: Nov. 28, 2002

(54) pH-indicator based assay for selective enzymes

(75) Inventors: Lana Elizabeth Janes, Toronto (CA); Christina A. Lowendahl, Lindome (SE); Romas J. Kazlauskas, Montreal (CA)

Correspondence Address: MCDONNELL BOEHNEN HULBERT & BERGHOFF, 300 SOUTH WACKER DRIVE, SUITE 3200, CHICAGO, IL, 60606, US

(73) Assignee: ThermoGen, Inc., Chicago, IL

(21) Appl. No.: 10/056,685

(22) Filed: Jan. 24, 2002

Related U.S. Application Data
(63) Continuation of application No. 09/344,885, filed on Jun. 25, 1999, now abandoned.
(60) Provisional application No. 60/090,569, filed on Jun. 25, 1998.

(51) Int. Cl. C12Q 1/34

(52) U.S. Cl. 435/18

(57) ABSTRACT

Provided herein is a method for the quantitative screening of hydrolase for desired substrate activity using pH indicators which are sensitive to the release of protons from a chemical reaction in a reaction mixture. The method comprises selecting buffer and indicator conditions such that both have the same affinity for protons such that the relative amount of buffer protonated is proportional to the amount of indicator protonated as the pH of the reaction mixture shifts. A reaction mixture is then prepared comprising a buffer, indicator, hydrolase to be tested, and desired substrate to be tested, allowing the hydrolase to react with the substrate. The reaction is monitored by detection of change in color of the reaction mixture, determined by the affect of the reaction on the indicator.

### RELATED APPLICATIONS

This application is a continuation-in-part of U.S. Ser. No. 60/090,569 filed Jun. 25, 1998.

### FIELD OF THE INVENTION

This invention relates to pH-dependent assays for enzyme activity using pH indicators and chromogenic substrates. The assay provides a method for identifying stereoselective enzymes using an efficient and rapid assay format.

### BACKGROUND OF THE INVENTION

Chemists often exploit the high stereoselectivity, chemoselectivity and regioselectivity of hydrolytic enzymes to solve synthetic problems. These reactions are often more selective, less costly and easier to carry out than chemical methods. To find a suitable hydrolase for a target compound, researchers first screen commercial enzymes and cultures of microorganisms and then optimize the reaction conditions. Both screening and optimization require measuring the selectivity of the reaction products.

One limitation to the wider use of hydrolases is the difficulty in finding the best hydrolase for a given reaction from hundreds of commercially available hydrolases and millions of microorganisms that express hydrolases. Several empirical rules are available to aid in the selection of likely candidates (see, for example, Kazlauskas, et al. J. Org. Chem. 1991, 56, 2656-2665; Franssen, et al. Tetrahedron: Asymmetry 1996, 7, 497-510; Chen, et al. J. Org. Chem. 1997, 62, 4349-4357). In addition, the majority of researchers also use screening. Screening typically includes running a small reaction for each hydrolase, “working-up” the reaction, and determining the ratio of stereoisomers using analytical methods such as high-performance liquid chromatography (HPLC), gas chromatography (GC) or nuclear magnetic resonance (NMR). Using equations developed by Sih et al. (Chen, et al. J. Am. Chem. Soc. 1982, 104, 7294-7299), the purity of the reaction products and/or percent conversion of the reaction are used to determine the selectivity. These are time consuming procedures, and usually result in incomplete screens since all hydrolases cannot be tested due to time constraints.

Researchers have previously utilized pH indicators to monitor the progress of enzyme-catalyzed reactions that release or consume protons. (Wajzer, M. J. C. R. Hebd. Seances Acad. Sci. 1949, 229, 1270-1272; R. A. John in Enzyme Assays, (Eds.: R. Eisenthal, M. J Danson), IRL, Oxford, 1992, pp. 81-82.). For example, researchers have monitored reactions catalyzed by amino acid decarboxylase (Rosenberg, et al. Anal. Biochem. 1989, 181, 59-65), carbonic anhydrase (Gibbons, et al. J. Biol. Chem. 1963, 238, 3502-3507), cholinesterase (Lowry, et al. J. Biol. Chem. 1954, 207, 19-37), hexokinase (Darrow, et al. Methods in Enzymology 1962, Vol. V, 226-235; Crane, R. K.; Sols, A. Methods in Enzymology, 1960, Vol I, 277-286) and proteases (Whittaker, et al. Anal. Biochem. 1994, 220, 238-243). Provided herein is a method for the identification of selective hydrolases, in particular enantioselective and diastereoselective hydrolases but not limited to this since this screen is also adaptable for regioselectivity.

Whittaker et al. (Anal. Biochem. 1994, 220, 238-243) measured the esterase activity of proteases in 96-well microplates using a pH-dependent assay, which shares certain features that are provided herein. However, the Whittaker assay requires additional calibration experiments because it does not use an indicator-buffer pair with the same pKa values and does not always accurately measure the true rates of enzyme-catalyzed hydrolysis. Thus, a sufficiently rapid and efficient method for identification of hydrolases using a color-based pH-dependent assay is not currently available.

Alternative methods to identify stereoselective hydrolases have been developed including measuring initial rates of hydrolysis for samples with varying ratios of enantiomers (Jongejan, et al. Recl. Trav. Chim. Pays-Bas 1991, 110, 247-254; van Tol, et al. Recl. Trav. Chim. Pays-Bas 1991, 110, 255-262) or by analyzing reaction progression curves (Lu, et al. Tetrahedron: Asymmetry 1995, 6, 1093-1096; Rakels, et al. Biotechnol. Bioeng. 1993, 43, 411-422; Fourneron, et al. Tetrahedron Letters 1992, 33, 2469-2472). However, such methods are not significantly faster and can be less accurate than the endpoint method. In addition, stereoselectivity has been previously estimated by separately measuring the rates of hydrolysis of the pure enantiomers (see, for example: Zandonella, et al. Chirality 1996, 8, 481-489; Reetz, et al. Angew. Chem. Int. Ed. Engl. 1997,36, 2830-2832).

The assay system described herein offers several advantages that are not provided by conventional screening methods. Provided herein is a rapid quantitative and colorimetric assay for identification of hydrolases using pH indicators. Using this method, stereoselectivity is determined by measuring the initial rates of hydrolysis for pure stereoisomers separately. Multiple hydrolases can be screened for stereoselectivity in a single series of rapid experiments. The method allows the investigator to quickly and accurately identify hydrolases having high stereoselective properties, providing both time and cost savings.

### SUMMARY OF THE INVENTION

Provided herein is a method for the quantitative screening of hydrolase for desired substrate activity using pH indicators which are sensitive to the release of protons from a chemical reaction in a reaction mixture. The method comprises selecting buffer and indicator conditions such that both have the same affinity for protons such that the relative amount of buffer protonated is proportional to the amount of indicator protonated as the pH of the reaction mixture shifts. A reaction mixture is then prepared comprising a buffer, indicator, hydrolase to be tested, and desired substrate to be tested, allowing the hydrolase to react with the substrate. The reaction is monitored by detection of change in color of the reaction mixture, determined by the affect of the reaction on the indicator.

### BRIEF DESCRIPTION OF THE DRAWINGS

FIG. 1. Schematic of the colorimetric screen for selectivity, in this example, enantioselectivity. The circles represent wells in a microplate containing either the (R)- or the (S)- enantiomers. Hydrolysis of an ester releases acid that decolorizes the pH indicator. Dark circles represent no reaction, while the white circles represent wells in which hydrolysis occurred. This diagram exaggerates the color change; in practice the color change is not always available to the naked eye. The hydrolase is rejected if neither the enantiomer reacts or if both enantiomers react at similar rates. If one enantiomer reacts significantly faster than the other, then the hydrolase is tested further. Note that measuring the rates of hydrolysis of pure enantiomers separately gives only an estimated enantioselectivity, not the true enantioselectivity.

FIG. 2. Sensitivity of the Assay Solution to Added Acid.

FIG. 3. Rates of Hydrolysis Measured by the Assay.

FIG. 4. (FIG. 5.4 from “Spec” paper) First step in the quick E measurement of enantioselectivity of a hydrolase toward (±)-4-nitrophenyl-2-phenylpropanoate, 1. Hydrolase-catalyzed hydrolysis of (S)-1 and resorufin tetradecanoate 6 releases yellow and pink chromophores, respectively. The solution turns a deep orange colour if both the substrates are hydrolyzed, pink if only the reference compound is hydrolyzed. The second step of the quick E is the same, except that it uses the (R)-enantiomer of the chiral ester. Equation 5.6 yields the selectivity ratio for each step. The extinction coefficients of both chromophores account for their partial ionization at pH 8; in practice, neither are fully ionized at pH 8.0 as shown above.

FIG. 5. Structures of chiral carboxylic acids for testing.

FIG. 6. Initial rates of hydrolysis of 4-nitrophenol esters are easily measured spectrophotometrically by measuring the linear increase in absorbance at 404 nm over time. The initial rate of hydrolysis of (S)-1 is determined in the first step to estimate enantioselectivity. The second step uses (R)-1 and the ratio of the two rates estimates the enantioselectivity. Note that the extinction coefficient accounts for the partial ionization of 4-nitrophenol at pH 7.5; in practice, 4-nitrophenol (pKa 7.15) is not fully ionized at pH 7.5 as shown.

FIG. 7. First Step Of The Quick E Measurement Of A Non-Chromogenic Ester, Solketal Butyrate 8, Using 4-Nitrophenol As The pH Indicator. Hydrolase-catalyzed hydrolysis of (S)-8 and resorufin acetate, 9, releases protons and the pink chromophore, resorufin. The rate of hydrolysis of resorufin acetate was calculated by the change in absorbance at 574 nm. To calculate rate of hydrolysis of (S)-8, we subtract the rate of protons released during hydrolysis of the reference compound from the total rate of protons detected with 4-nitrophenol. The ratio of relative rates, (S)-8/9 is the selectivity ratio for the first step in quick E. The second step is the same but uses the (R)-enantiomer and resorufin acetate. The ratio of selectivity ratios from both steps yields quick E.

FIG. 8. Measuring Selectivity of Hydrolases. a) For estimated selectivities, the initial rate of hydrolysis of different esters is measured colorimetrically using 4-nitrophenol as a pH indicator. b) Quantitative measure of selectivity requires a competitive experiment. Resorufin acetate was used as a competitive substrate because its hydrolysis generates the easily measured resorufin anion. The total amount of hydrolysis is measured using 4-nitrophenol as the pH indicator as in part a. Only 10% of the resorufin is deprotonated to the anion at pH 7.2, thus hydrolysis of one mole of resorufin generates 1.1 mole of protons.

FIG. 9. Esters Used to Survey _{ThermoGen} esterases.

FIG. 10. Enantiomer Pairs Used to Survey Enantioselectivity of _{ThermoGen} esterases.

### DETAILED DESCRIPTION

The present invention relates to assays for enzyme activity by detection of proton release during hydrolysis reactions with a pH indicator. All references cited herein are incorporated by reference.

A current limitation in the identification of hydrolases is finding the optimal hydrolase for a particular application. The most utilized of the currently available systems for this purpose is the “endpoint method” of Sih and coworkers (Chen, et al. J. Am. Chem. Soc. 1982, 104, 7294-7299). However, the method of Sih can be tedious and time-consuming. Recognizing a need for a faster approach, researchers have proposed other methods (see, for example Jongejan et al. Recd. Trav. Chim. Pays-Bas 1991, 110, 247-254; Fourneron et al. Tetrahedron Letters, 1992, 33, 2469-2472), but these can also lack the efficiency and accuracy required to simply perform a comprehensive assay for stereoselective hydrolases.

Provided herein is a method for the identification of stereospecific hydrolases using pH indicators and reference compounds, amenable to both single assay and high-throughput formats. In a preferred embodiment, a method for identification of stereoselective hydrolases is provided. In another preferred embodiment, a method for identification of regioselective hydrolases is provided.

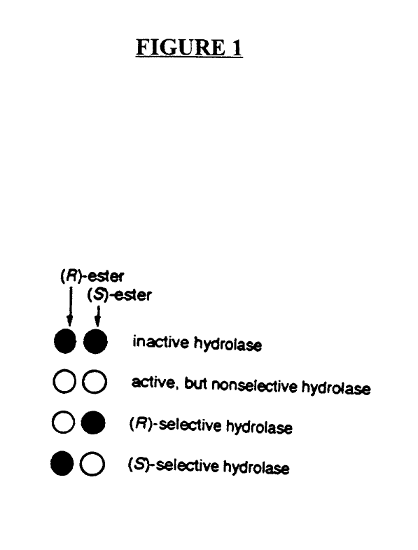

There are several advantages provided by the instant invention that are not provided by conventional screening methods. For instance, the instant assay system provides useful data much more quickly and efficiently that conventional screening methods. The assay is based on detection of color change which is ordinarily not detected to the naked eye (FIG. 1).

Another advantage is that the instant method is quantitative, unlike conventional methods used to screen for hydrolytic activity such as thin layer chromatography (TLC). And, the available multi-well format allows the analysis of large numbers of samples simultaneously. As the entire reaction and analysis typically occurs in a microplate well, additional laborious workup and analysis by gas chromatography (GC), high-perfornance liquid chromatography (HPLC) or nuclear magnetic resonance (NMR) is avoided.

Yet another advantage is that the instant invention requires significantly less substrate (typically 20 μg in 360 μl) and test enzyme (sometimes less than 1 μg protein). The amount of enzyme required will vary depending on the particular assay, and may be more or less than 20 μg/well. As such, the instant assay is useful for screening libraries of mutant enzymes obtained using recombinant DNA methods, such as directed evolution technologies that utilize error-prone polymerase chain reaction (PCR) or DNA shuffling technologies.

Another advantage is that this assay can measure the hydrolysis of any ester, not just esters that incorporate a chromophore in their structure. The ability to screen the target ester itself, and not an analog, is an important advantage since an enzyme's selectivity is sensitive to small changes in substrate structure.

Basic Parameters of the Stereoselective Assay

Hydrolysis of an ester at neutral pH releases a proton. Hydrolysis of an exemplary ester, solketal butyrate (butyryl ester of 2,2-dimethyl-1,3-dioxolane-4-methanol) is shown below.

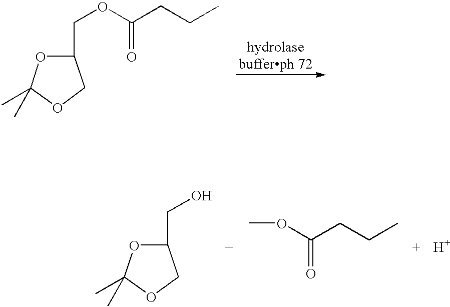

The present invention provides a method for measuring the rate of proton release using a pH indicator as illustrated below. By choosing the reaction conditions carefully, one can ensure that the color change is proportional to the number of protons which, in turn, relates to the rate of hydrolysis of the substrate.

The basic assay requires a buffer, a pH indicator, a substrate compound and an enzyme to be assayed for stereoselectivity. Hydrolysis of the substrate compound causes a color change in the solution by allowing for the protonation of the pH indicator, whose extinction coefficient changes upon protonation. Typically, the assay is performed in a multi-well reaction vessel.

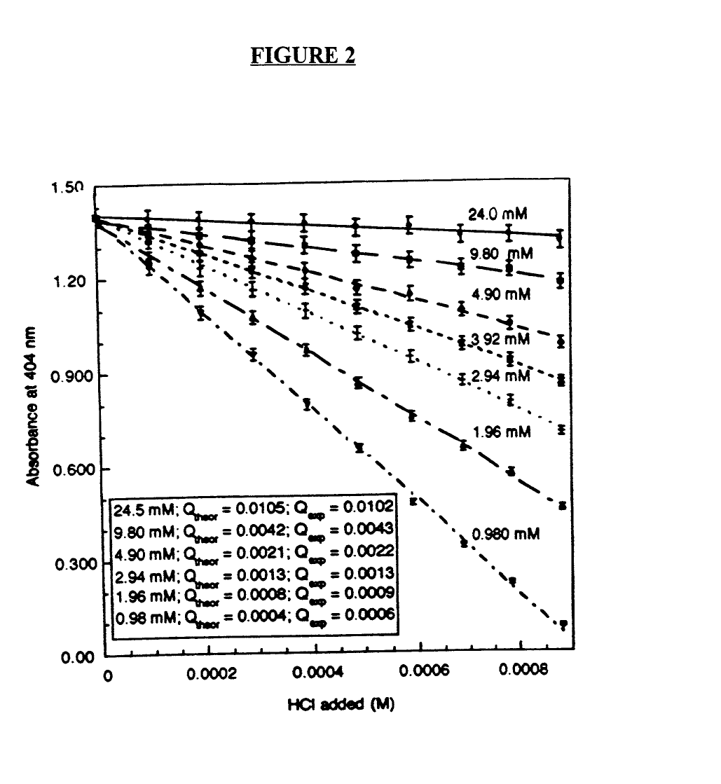

In practicing the present invention, many suitable buffers and indicators are available to the skilled artisan. It is preferable that both the buffer and the indicator have approximately the same affinity for protons (pKₐ buffer=pKₐ indicator). For the purposes of this application, a pKₐ is said to be “identical” or “the same” as another pKₐ if it is within 0.1 units of the other pKₐ. For instance, if the pKₐ of Buffer 1 is 7.0 and the pKₐ of Indicator 1 is 7.1, the pKₐ's are understood to be identical or the same. It is preferable that the buffer and the indicator each have a pKₐ within 0.1 unit of each other so that the relative amount of protonated buffer and protonated indicator remains constant as the pH shifts during the reaction. In one calculation, a difference in pKₐ between the buffer and the indicator of 0.3 units typically resulted in an approximate 8% error when the pH changes by 0.1 unit. In a typical assay, the pH may change by, for example, 0.05 units (10% hydrolysis of the substrate); thus, differences in pKₐ can lead to non-linear and inaccurate rates. If a different pKₐ between the buffer and the indicator cannot be avoided, accurate results may still be obtained using calibration experiments or a more complex equation. As a simple test, an approximately linear relationship between an amount of standard acid added to the assay solution containing a suitable matching pH-indicator and buffer and the measured color change indicates that that the pKₐ of the buffer and the indicator are within the preferable range (see, for example FIG. 2, discussed below). Further, the agreement of the theoretical and experimental slopes to within about 5% establishes that the assay is quantitative. It is well within the skills of the ordinary skilled artisan to make such determinations. It is to be understood that buffer-indicator combinations where the buffer and the indicator have different pKₐ values (ie, greater than a 0.1 unit difference) are encompassed by the instant invention.

The skilled artisan may use any suitable buffer in practicing the present invention. Many such suitable buffers are available to the skilled artisan. Suitable buffers for screening at certain pH ranges may include but are not limited to MES (2-[N-morpholino]ethanesulfonic acid, pKₐ 6.1; useful for screening at a pH of approximately 6), BES (N,N-bis[2-hydroxyethyl]-2-aminoethanesulfonic acid, pKₐ 7.15; useful for screening at a pH of approximately 7), EPPS (N-[2-hydroxylethyl]piperazine-N′-[3-propanesulfonic acid], pKₐ 8.0; useful for screening at a pH of approximately 8), and CHES (2-[N-cyclohexylamino]ethane-sulfonic acid, pKₐ 9.3; useful for screening at a pH of approximately 9). The skilled artisan would be aware of many other such suitable buffers that may utilized in practicing the present invention, certain of which may be listed in commonly utilized technical references such as Beynon, et al. (Buffer Solutions, The Basics, IRL Press, Oxford, 1996).

There is also a wide array of suitable pH indicators available to the skilled artisan. Preferably, the protonated and deprotonated forms of the pH indicator have large differences in extinction coefficients (for example, 200 vs 18,000 M⁻¹ cm⁻¹ at 404 nm for 4-nitrophenol) which allows for good sensitivity. Suitable pH indicators include but are not limited to chlorophenol red (pKₐ 6.0) useful for screening at a pH of approximately 6, 4-nitrophenol (pKₐ 7.2) useful for screening at a pH of approximately 7, phenol red (pKₐ 8.0) useful for screening at a pH of approximately 8, and thymol blue (pKₐ 9.2) useful for screening at a pH of approximately 9. Other suitable indicators may be identified in the Merck Index (The Merck Index, 10th ed., Merck & Co., Rahway, N.J., 1983), Indicators (ed. By E. Bishop, Pergamon Press, New York, 1972), or the Sigma/Aldrich Handbook related to dyes and indicators (The Sigma-Aldrich Handbook of Stains, Dyes and Indicators' by Floyd J. Green, published by Aldrich Chemical Company in 1990, Milwaukee, Wis.). In addition, the skilled artisan would be aware of many other such suitable pH indicators that may be utilized in practicing the present invention.

Those skilled in the art would recognize that the present assay could be utilized to assay enzyme activity at many different pH ranges utilizing specific pH indicator/buffer combinations. As the majority of hydrolases have maximal activity near neutral pH, an assay designed for use at pH 7.2 could use, for example, 4-nitrophenol as a pH indicator due to the similarity of its pKₐ (7.15) (The Merck Index, 10th ed., Merck & Co., Rahway, N.J., 1983, p. 950.) If the pKₐ of an indicator is not known, the pKₐ could be ascertained by measuring the midpoint of the pH change as standardized base is added. It should also be understood that the pKₐ may shift under different assay conditions, for example, cosolvent and ionic strength may change the pKₐ values and that the skilled artisan should take this into account.

Exemplary buffer-indicator combinations include but are not limited to the following: chlorophenol red (pKₐ 6.0) and MES (2-[N-morpholino]ethanesulfonic acid, pKₐ 6.1) for use at a pH of approximately 6; 4-nitrophenol (pKₐ 7.2) and BES (N,N-bis[2-hydroxyethyl]-2-aminoethanesulfonic acid, pKₐ 7.15) for use at a pH of approximately 7; phenol red (pKₐ 8.0) and EPPS (N-[2-hydroxylethyl]piperazine-N′-[3-propanesulfonic acid], pKₐ 8.0) for use at a pH of approximately 8; thymol blue (pKₐ 9.2) and, CHES (2-[N-cyclohexylamino]ethane-sulfonic acid, pKₐ 9.3) for use at a pH of approximately 9. In a preferred embodiment, the buffer-indicator combination is BES (N,N-bis[2-hydroxyethyl]-2-aminoethanesulfonic acid) and 4-nitrophenol (pKₐ 7.2) because the pKₐ of BES (7.15) (Beynon, et al. Buffer Solutions, The Basics, IRL Press, Oxford, 1996, p. 72) is the same as that of 4-nitrophenol. The skilled artisan would be aware of many other such suitable buffer-pH indicator combinations that may utilized in practicing the present invention.

The optimal buffer concentration should represent a compromise between low concentration to maximize sensitivity, (see Eq. 1) and small pH changes throughout the assay (<0.05 pH units for 10% hydrolysis at our conditions). The small pH changes are important because kinetic constants can change with changing pH.

The concentration of the pH indicator should be as high as possible to maximize sensitivity, see Eq. 1. The pathlength in a 96-well plate depends on the volume of the solution in the well since the light passes from the top of the plate through the solution. Thus, the maximum indicator concentration varies with the solution volumes and also with the extinction coefficient of the pH indicator. With certain acid-base indicators, poor water solubility can limit the maximal concentration of the pH-indicator. It is preferable to include a control sample in the assay to ensure there is no enzyme inhibition by the indicator.

The extinction coefficients change slightly upon addition of cosolvent and should be determined experimentally. The experimental result must ensure that upon addition of a cosolvent to the reaction mixture, the pH changes result in a detectable color change. For instance, it is preferable that the assay tolerate small changes in reaction conditions, such as the addition of 7% v/v acetonitrile or other organic solvent. For instance, it has been previously shown that the pKₐ of 4-nitrophenol changes only slightly from 7.15 to 7.17 upon addition of 10% ethanol. It is preferable that co-solvent concentrations below 10% do not compromise the accuracy of the assay. It is further preferable that small amounts of salts present in the hydrolase solutions (buffer salts in commercial hydrolase preparations, CaCl₂ in the protease solutions) do not affect the accuracy.

Many suitable substrate concentrations may be utilized in practicing the present invention. Preferably substrate concentrations range from 0.5 to 2 mM, and are more preferably approximately 1 mM. At substrate concentration below about 5.0 mM, the absorbance changes may be too small to be detected accurately using approximately 0.5 mM buffer and 0.5 mM indicator. For example, hydrolysis of 5% of a 0.25 mM substrate concentration at pH 7.2, 0.45 mM 4-nitrophenol, 5 mM BES may change the absorbance by only 0.005 absorbance units. Under lower substrate concentrations, lower buffer concentrations can be utilized to maximize the change in absorbance. Solubility in water sets the upper limit of substrate concentration because spectrophotometric measurements require stable solutions and it is preferable that the solutions are clear. Typical organic substrates dissolve poorly in water and organic cosolvent may be added (ie, 7 vol % acetonitrile). For very insoluble substrates, clear emulsions using detergents may be utilized (Janes, et al. J. Org. Chem. 1997, 62, 4560-4561).

The present invention could be utilized in any suitable reaction vessel, and examples of such vessels are well known to those skilled in the art. Suitable reaction vessels may include but are not limited to polystyrene or polypropylene vessels having a sufficient number of wells, the number of which can be determined by the skilled artisan. In one embodiment, the reaction vessel has 96 wells. In another embodiment, the reaction vessel has 384 wells. In yet another embodiment, the reaction vessel has more than 384 wells. Individual cuvettes can also be utilized.

Additional Parameters of the Assay

Certain formulas are important to a description of the mechanics of the instantly provided assay. The proportionality between the rate of indicator absorbance change and reaction rate is commonly referred to as the buffer factor, Q (Rosenberg, et al. Anal. Biochem. 1989, 181, 59-65; Gibbons, et al. J. Biol. Chem. 1963, 238, 3502-3507). When the pKₐ of the indicator and buffer are the same, Q is given by Eq. 1 where C represents the total molar concentration (sum of acid and base forms) of buffer (B) or indicator (In), “Δε” represents the difference in extinction coefficient between the protonated and deprotonated forms of the indicator and “1” represents the path length:

$$Q = \frac{{C}_{B}}{{C}_{I n}} \times \frac{1}{Δ ϵ \times 1} \qquad ( Eq . 1 )$$

The true reaction rate is given by Eq. 2, where dA/dt is the rate of indicator absorbance change. The highest sensitivity (ie, largest dA/dt) occurs when Q is small. Thus, lowering the buffer concentration or increasing the indicator concentration increases the sensitivity of the assay.

$$Rate ( µ mol \text{/} min ) = \frac{ⅆ A}{ⅆ t} \times Q \times reaction volume \times {10}^{6} \qquad ( Eq . 2 )$$

The rates of hydrolysis of both stereoisomers (in this example, enantiomers) of a pair can be measured separately and the ratio of initial rates estimates the stereoselectivity. This is a rapid and useful method to estimate enantioselectivity as shown below:

$$Estimated Enantioselectivity = \frac{rate of fast enantiomer}{rate of slow enantiomer}$$

However, the above equation only estimates the stereoselectivity, in the above example, enantioselectivity. This equation can be adapted for estimated diastereoselectivity.

The enantioselectivity of an enzyme is the ration of rates of hydrolysis of the two enantiomers in solution together. It can also be expressed as the ratio of the specificity constants (K_{cat}/K_{M}) for the enantiomers (see Eq. 3) (see also, generally, Fersht, A. Enzyme Structure and Mechanism, 2nd ed., Freeman: New York, 1985, pp 103-106; Chen, et al. J. Am. Chem. Soc. 1982, 104, 7294-7299).

$$Enantiomeric ratio = E = \frac{{v}_{fast}}{{v}_{slow}} = \frac{{( {k}_{cat} / {K}_{M} )}_{fast enantiomer}}{{( {k}_{cat} / {K}_{M} )}_{slow enantiomer}} \qquad ( Eq . 3 )$$

When initial rates are measured under conditions where the pure enantiomer substrate concentration is below its K_{M} value, then the initial rate is equal to its k_{cat} and K_{M} value following simple Michaelis-Menton equations. When the substrate concentration is above its K_{M}, the initial rate is equal to its k_{cat}. Enantioselectivity is the ratio of k_{cat}/K_{M} for both enantiomers, so estimated E can ignore some or all of the effects of K_{M} on enantioselectivity.

Currently, the most useful method for measuring E is the endpoint method developed by C. J. Sih's group (Chen, et al. Quantitative Analyses of Biochemical Kinetic Resolutions of Enantiomers, J. Am. Chem. Soc. 1982, 104, 7294-7299), but screening hundreds of commercial enzymes or cultures of microorganisms by this method is difficult. To measure E, the skilled artisan typically runs a test resolution, works up the reaction, and measures two of the following: enantiomeric purity of the starting material (eeₛ), enantiomeric purity of the product (eeₚ), or conversion (c). They then use the integrated forms of Eq. 3 developed by Sih and coworkers, which relates to the degree of conversion of the reaction to the enantiomeric purity of the remaining substrate (eeₛ) or resulting product (eeₚ), Eq. 4.

$$E = \frac{ln [ ( 1 - c ) ( 1 - e {e}_{s} ) ]}{ln [ ( 1 - c ) ( 1 + e {e}_{s} ) ]} = \frac{ln [ ( 1 - c ) ( 1 + e {e}_{p} ) ]}{ln [ ( 1 - c ) ( 1 - e {e}_{p} ) ]} = \frac{ln ( 1 - e {e}_{s} ) / 1 + \frac{e {e}_{s}}{e {e}_{p}}}{ln ( 1 + e {e}_{s} ) / 1 + \frac{e {e}_{s}}{e {e}_{p}}} \qquad ( Eq . 4 )$$

A typical assay for measuring the enantioselectivity of a hydrolase towards a substrate, using conventional methods, requires up to four and a half hours to complete. For instance, the skilled artisan may first carry out the kinetic resolution to 40% conversion (approx. 2 h), separate remaining substrate from the product acid (approx. 30 min), hydrolyse unreacted substrate to the acid with an aqueous base such as NaOH (approx. 1 h including workup) and finally measure the enantiomeric purity of both samples by HPLC on a chiral stationary phase (approx. 1 h). This costs the investigator great amounts of time and can result in decisions to test limited numbers of enzymes, resulting in a less than complete analysis.

Provided herein is the novel spectrophotometric method (termed “Quick E”) that is an extension of a pH indicator method to accurately measure the enantioselectivity of hydrolases based upon the measurement of initial rates. Rather than measure the relative rates of hydrolysis of a racemic solution to determine E (this would require measuring enantiomeric purity to evaluate the relative rate of hydrolysis of each enantiomer), the rates of hydrolysis of each enantiomers relative to a reference compound are separately measured. A specificity ratio (k_{cat}/K_{M}) is then obtained for each pure enantiomer relative to the reference compound. The ratio of the relative specificity constants for each pure enantiomer yields the enantioselectivity. (k_{cat}/K_{M})fast enantiomer/(k_{cat}/K_{M})slow enantiomer, Eq. 3.

In the simple case of a hydrolase-catalyzed reaction, the observed initial rate (νᵢₙᵢₜᵢₐₗ) of a hydrolysis of a substrate (S) can be expressed by the following steady-state equation (Eq. 5) where [E] is the concentration of free enzyme and [S] is the initial concentration of ester. It should be noted that these equations assume Michealis Menten kinetics and are under certain limitations described below.

$$v = \frac{ⅆ [ S ]}{ⅆ t} = ( \frac{{k}_{cat}}{{K}_{M}} ) \times [ S ] \times [ E ] \qquad ( Eq . 5 )$$

When two substrates are present in solution, they both compete for the enzyme's active site. From Eq. 5, it follows that the ratio of rates of hydrolysis equals the ratio of their k_{cat}/K_{M} values (the specificity constants), after taking into account the concentration of both substrates. If the two substrates are a pair of enantiomers, the ratio of their specificity constants equals the enantiomeric ratio, E, Eq. 3.

The traditional method to determine k_{cat} and K_{M} measures the initial rates of hydrolysis of each enantiomer as a function of its substrate concentration is measured and the data is transformed into a linear form for analysis. (See generally, Fersht, A. Enzyme Structure and Mechanism, 2nd ed., Freeman: New York, 1985, pp 103-106; Chen, et al. J. Am. Chem. Soc. 1982, 104, 7294-7299). Although this method reveals useful information on the kinetics of each enantiomer as well as the overall enantioselectivity, it is unsuitable for screening large numbers of hydrolases for enantioselectivity. It is time-consuming because it requires multiple measurements for each pure enantiomer and significant data analysis. The method provided herein for measuring relative k_{cat} and K_{M} values is faster, requiring at a minimum a single measurement for each enantiomer. Other researchers have previously used mixtures of substrates to measure enzyme selectivity towards mixtures of substrates (Berman, et al. J. Biol. Chem. 1992, 267, 1434-1437; Birkett, et al. J. Anal. Biochem. 1991, 196, 137-143; Petithorny, et al. Proc. Natl. Acad. Sci. USA 1991, 88, 11510-11514; Schellenberger, et al. Biochemistry 1993, 32, 4344-4348). This method is used to measure the stereoselectivity of an enzyme.

In one embodiment, the present invention provides the “quick E” method, which provides the skilled artisan with several distinct advantages over conventional assays. The quick E method is many times faster than a typical endpoint measurement, yet can have equivalent or better accuracy. Accuracy may be particularly important for screening techniques related to directed evolution experiments where the improvements of each generation are small (Moore, et al. Nature Biotechnology 1996, 14,458-468). Furthermore, the Quick E method requires much smaller amounts of hydrolase because the entire reaction occurs in the spectrophotometer.

Quick E is based on the same equations as the endpoint method, so inaccuracies of the endpoint method also apply to quick E. Due to assumptions made in deriving Eq. 4, both the erdpoint method and quick E may result in inaccurate enantioselectivites where the reaction includes an impure biocatalyst or the reaction is inhibited by product. First, E values calculated using impure biocatalyst are a weighted average of all the enzymes. If these enzymes differ significantly in their affinity for the substrate, then different enzymes will dominate the activity at different substrate concentrations. And, when product inhibits the reaction, the apparent renantioselectivity may change. To include product inhibition in the quantitative analysis, reseachers use more complex equations which take into account the mechanism of lipase-catalyzed ping-pong bi-bi reactions (Rakels, et al. Biotechnol. Prog. 1994, 10, 403-409; van Tol, et al. Biocatal. Biotransform. 1995, 12, 119-136). The deviations are usually small, so that researchers consider them only in the final process optinization.

Thus, an assay system is provided wherein the stereoselectivity of an enzyme can be determined rapidly and with a high degree of accuracy. This assay provides the skilled artisan with a valuable tool for identification of stereoselective enzymes. The following examples are for illustrative purposes only and are not intended, nor should they be construed as limiting the invention in any manner. Those skilled in the art will appreciate that variations and modifications can be made without violating the spirit or scope of the invention.

### EXAMPLES

### Example 1

A. Materials and Methods

1. Materials

Chemicals were purchased from Sigma Chemical Co. (Oakville, ON) and were used without further purification unless stated. Triton X-100 was purchased from ESA Inc. (Chelmesford, Mass.). Standardized acid was purchased from A & C American Chemicals Ltd. (Montréal, QC). EDC-HCl (N-ethyl-N′-[3-(dimethylamino) propyl]carbodiimide hydrochloride) and 1-hydroxybenzotriazole (anhydrous) were purchased from Chem-Impex Int. (Wood Dale, Ill.). TRIS buffer was purchased from ICN Biomedicals, Inc. (Aurora, Ohio). Unless otherwise noted, esters were purchased from Aldrich, Fluka, or TCI. Solketal butyrate was prepared previously. Solketal octanoate and phenethyl butyrate were prepared similarly. _{ThermGen} esterases were obtained from _{ThermoGen}, Inc. (Chicago, Ill.). Acetyl esterase from orange peel was purchased from Sigma.

Lipase was purified by the method of Colton et. al. Crude Candida rugosa lipase (20 g. solid, 800 units by PNPA assay) was dissolved in MES buffer (100 mL, 50 mM, pH 6.0, 4° C.) by stiring for 30 minutes. Isopropanol (100 mL, 4° C.) was added dropwise over 30 minutes and allowed to stir for 48 h at 4° C. The solution turned from clear to cloudy while stirring. A precipitate was removed by centrifuging at 3000 rpm for 30 min at 4° C. The supernatant was dialyzed against doubly distilled water (3×4 L) and concentrated to 20 mL by ultrafiltration under N₂ using an Amicon PM-10 membrane: 692 units with PNPA assay, 16.8 mg protein by the Bio-Rad protein assay using BSA as the standard, 87% yield. The clear yellow enzyme solution was stored at 4° C. with 0.02% wt/vol % NaN₃ as preservative.

The pure enantiomers and racemate of 2-(4isobutylphenyl)propanoic acid were obtained from the Biotechnology Research Institute (Montreal, Quebec). Enzyme suppliers are noted in the footnotes of Table 1.

Polystyrene 96-well flat-bottomed microplates (maximum volume 360 μl/well, Corning Costar, Acton, Mass.) were filled using Eppendorff 8-channel pipettes (5-100 μl, 50-1,200 μl) and solution basins for multichannel pipettes (Fisher Scientific, Nepean, ON). All microplate assays were performed on a Spectramax 340 microplate reader with SOFTmax PRO version 1.2.0 software (Molecular Devices, Sunnyvale, Calif.).

The initial rates of small-scale enzyme-catalyzed hydrolysis reactions were measured with a Radiometer RTS 822 pH stat. NMR were recorded on a Varian Gemini 200 MHz spectrometer. Melting points were taken on an Electrothermal melting point apparatus and were corrected.

Mass spectra were acquired using EI (70 eV) conditions or CI (NH3 as ionization gas) on a Kratos MS25RFA double focussing mass spectrometer.

High performance liquid chromatography (HPLC) was performed on a Spectra Physics liquid chromatograph, model 8800, equipped with a Spectra FOCUS forward optical scanning detector, SP8800 autosarnpler and Spectra Physics software. HPLC chiral stationary phases were purchased from Daicel Chemical Industries Ltd. (Fort Lee, N.J.).

2. (±)-Solketal Butyrate

Butyric anhydride (1.5 equiv.), 4-dimethylaminopyridine (0.05 equiv.) and anhydrous sodium carbonate (1.5 equiv.) were added to a solution of (±)solketal (1.0 equiv.) in ethyl acetate and stirred overnight. The reaction mixture was washed several times with water, then with brine, and the organic extract was dried with magnesium sulfate. Flash chromatography (3:1 hexanes:ethyl acetate) afforded the pure butyryl ester as a yellow oil in 91% yield. R_{f}=0.56 (3:1 hexanes:ethyl acetate), ¹H-NMR (CDCl3, 200 MHz) δ=0.98 (t, 3J (H,H)=7.4 Hz, 3H, CH₃), 1.37 (s, 3H, CH₃), 1.43 (s, 3H, CH₃), 1.67 (sextet, 3J (H,H)=7.4 Hz, 2H, CH2), 2.33 (t, 3J (H,H) =7.3 Hz, 2H, CH₂), 3.7 (m, 1 H of CH₂), 4.05-4.16 (m, 3H, 1 H of CH₂, CH₂), 4.27-4.32 (m, 1H, CH). ¹³C-NMR (CDCl3, 200 MHz) δ=15.3 (CH₃), 19.9 (CH₂), 26.9 (CH₃), 28.1 (CH₃), 37.3 (CH₂), 65.5 (CH₂), 67.3 (CH₂), 74.6 (CH), 110.3 (C), 173.1 (C═O).

3. (S)-Solketal Butyrate and (R)-solketal Butyrate

Samples were prepared from enantiomerically pure solketal as above for the racemate. The enantiomeric purities of the butyrates measured by GC (see below) were 99.2% and 99.8%, respectively. No contaminating butyric acid or solketal were detected by GC or ¹H-NMR.

4. Synthesis of Substrates

All other esters were synthesized using a modified DCC coupling employing a water soluble coupling reagent (EDC-HCl; N-ethyl-N′-[3-(dimethylamino) propyl]carbodiimide hydrochloride) unless stated otherwise. Alcohol (1.1 equiv.), acid and 1-hydroxybenzotriazole (1.1 equiv.) were added to anhydrous dichloromethane in a round bottom flask stirring in an ice bath. The mixtures stirred for 15 minutes, then EDC-HCl (1.1 equiv.) was added. The reactions were allowed to warm to room temperature and stirred for 48 hours, followed by washings with saturated sodium bicarbonate, water, 1 N HCl, then water, in that order. Esters were purified by silica gel chromatography eluted with hexanes/ethyl acetate and recrystallized, when solids, from hexaneslethyl acetate, unless stated otherwise.

(±)-4-nitrophenyl-2-phenylpropanoate. 1. 4-nitrophenol was recrystallized from chloroform and (±)-2-phenylpropanoic acid was purified by vacuum distillation prior to coupling. The reaction afforded 2.35 g (10.4 mmol, 78% yield) of the title compound as a yellow solid: R_{f}=0.53 (3:1 hexanes:ethyl acetate); mp=155.8-162.5° C.; ¹H-NMR (200 MHz, CDCl3) 1.64 (d, 3H, J=7.0 Hz), 3.96 (q, 1H, J=7.2 Hz), 7.18 (d, 2H, J=9.0 Hz), 7.38 (m, 5H), 8.23 (d, 2H, J=9.2 Hz); ¹³C-NMR (200 MHz, CDCl3) δ19.9, 46.9, 122.6, 125.4, 127.7, 128.0, 129.2, 139.5, 145.4, 155.5, 171.8; MS (EI) m/z: 271 (M·+, 5); 133 (59 d 1.64 (d, 3H, J=7.0 Hz), 3.96 (q, 1H, J=7.2 Hz), 7.18 (d, 2H, J=9.0 Hz), 7.38 (m, 5H), 8.23 (d, 2H, J=9.2 Hz); ¹³C-NMR (200 MHz, CDCl3) δ19.9, 46.9, 122.6, 125.4, 127.7, 128.0, 129.2, 139.5, 145.4, 155.5, 171.8; MS (EI) m/z: 271 (M″+, 5); 133 (59), 105 (100), 103 (4), 79 (4), 77 (4), 63 (1), 51 (1); HRMS (EI): calcd. for C₁₅H₁₃NO₄: 271.08460; found: 271.08445, 0.6 ppm error.

(R) and (S)-4-nitrophenyl-2-phenylpropanoate, (R) and (S)-1. Samples were prepared from enantiomerically-pure 2-phenylpropionic acid as above for the racemate. The enantiomeric purities of the esters measured by HPLC (see below) were 99.7% and 99.4%, respectively. Yields for the reactions were 51% and 56%, respectively.

(±)-4-nitrophenyl 2-(4-isobutylphenyl)propanoate 2. The reaction afforded 0.70 g (2.14 mmol, 44% yield) of the product as a yellow solid after silica gel chromatography with 100% chloroform as eluent, followed by recrystallization: Rf=0.57 (chloroformmp=57.8-60.0° C.; ¹H-NMR (200 Mz, CDCl3) δ0.917 (d, 6H, J =6.6 Hz), 1.63 (d, 3H, J=7.2 Hz), 1.91 (m, 1H), 2.48 (d, 2H, J=7.0 Hz), 3.96 (q, 1H, J=7.2 Hz), 7.14-7.31 (m, 6H), 8.23 (d, 2H, J=9.2 Hz); ¹³C-NMR (200 MHz, CDCl3) 19.9, 23.9, 31.6, 46.3, 46.5, 111.4, 122.6, 125.4, 127.4, 129.9, 136.6, 141.3, 172.0; MS (EI) m/z: 327 (Me·+, 6), 189 (3), 161 (100), 145 (4), 117 (11.2), 105 (3), 91 (5); HRMS (EI): calcd. for C₁₉H₂₁NO₄: 327.14720; found: 327.14705,0.5 ppm error.

(R)-and (S)-4-nitrophenyl 2-(4-isobutylphenylpropanoate, (R) and (S)-2. Samples were prepared from enantiomerically-pure 2-(4-isobutylphenyl)propanoic acid as above for the racemate. The enantiomeric purities of the esters measured by HPLC (see below) were 98.2% and 99.6%, respectively, after hydrolysis to the acid using aqueous NaOH for analysis. Yields for the reactions were 68 and 65%, respectively.

D,L-phenylalanine-4-nitrophenol ester TFA salt 3. D,L-phenylalanine was converted to its N-t-BOC derivative using di-tertbutyl-pyrocarbonate following a procedure of Tarbell et al. The protected amino acid was then converted to its 4-nitrophenyl ester following the coupling procedure above. The t-BOC group was subsequently removed by stirring the solid compound in 5 mL neat TFA for 10 minutes, followed by removal of TFA in vacuo. The final product was recrystallized from chloroform affording the title compound as a white, fluffy solid in 45% yield overall from three steps: mp=177.1-178.9° C. (sample darkens at 160° C.); ¹H-NMR (200 MHz, CD3OD) 3.41 (d, 2H, J=7.2 HZ), 4.68 (t, 1H, J=7.2 Hz), 7.29 (d, 2H, J =9.4 Hz), 7.32-7.43 (mM, 5H), 8.36 (d, 2H, J=9.2 Hz); MS (EI) m/z: 286 (Me·+, 2), 240 (2), 195 (23), 167 (4), 120 (100), 91 (29), 69 (19), 46 (23); HRMS (EI): calcd. for C₁₅H₁₄N₂O₄: 286.93;.09536; found: 286.09520, 0.5 ppm error.

D- and L-phenylalanine-4-nitrophenol ester TFA salt, D and L-3. Samples were prepared from enantiomerically-pure L- and D- N-t-BOC-phenylalanine-4-nitrophenol ester (Sigma-Aldrich Co., 99% purity) by removal of the N-t-BOC group as for the racemate. The enantiomeric purities of the esters measured by HPLC (see below) were both >99.5% (see below).

5. Synthesis of Reference Compounds

Resorufin acetate 9. This compound was prepared using a modified procedure by Kramer and Guilbault. To a slurry of resorufin sodium salt (95% purity, 1.015 g, 4.3 mmol, 1 eq.) in 60 mL anhydrous dichloromethane, was added anhydrous pyridine (0.349 mL, 4.3 mmol, 1 eq). The solution was cooled in an ice bath, then acetyl chloride (0.614 mL, 8.6 mmol, 2 eq) was added dropwise over 10 minutes. The deep purple reaction mixture immediately turned orange. The reaction was warmed to room temperature and stirred overnight. The reaction was next diluted with dichloromethane to 300 mL and filtered through a coarse glass frit to remove unreacted resorufin, and the solvent removed in vacuuo. The reddish-orange residue was recrystallized from ethanol yielded 0.48 g (1.89 mmol, 44% yield). of a crimson powder: R_{f}=0.20 (2:1 hexanes:ethyl acetate); mp=217.4-220.2° C. (sample darkens at 215.4° C.) [literature=223-225° C. (uncorrected)]; ¹H-NMR (CDCl₃, 200 MHz) δ2.37 (s, 3H), 6.34 (d, 1H, J=1.8Hz), 6.90-6.84 (dd, 1 H, J=2.1 Hz, 9.9 Hz), 7.11-7.16 (dd, 1H, J=2.2 Hz, 7.3 Hz), 7.16 (s, superimposed, 1H), 7.44 (d, 1H, J=9.7 Hz), 7.81 (d, 1H, J=4.4 Hz); ¹³C-NMR (200 MHz, D6-DMSO) δ22.2, 106.3, 110.1, 119.7, 130.8, 130.9, 134.6, 135.0, 143.8, 147.9, 149.2, 152.9, 168.2, 184.9; MS (EI) m/z: 255 (M·+, 14); 213 (100); 185.16 (dd, 1H, J=2.2 Hz, 7.3 Hz), 7.16 (s, superimposed, 1H), 7.44 (d, 1H, J=9.7 Hz), 7.81 (d, 1H, J=4.4 Hz); ¹³C-NMR (200 MHz, D6-DMSO) δ22.2, 106.3, 110.1, 119.7, 130.8, 130.9, 134.6, 135.0, 143.8, 147.9, 149.2, 152.9, 168.2, 184.9; MS (EI) m/z: 255 (M″+, 14); 213 (100); 185 (72); 156 (7); 128 (4); 63 (14); 43 (9). HRMS (EI): calcd. for C14H9N1O4: 255.05315; found: 255.05330, 0.6 ppm error.

Resorufin tetradecanoate 6. The procedure was similar to the acetate derivative but myristic anhydride was added dropwise over 10 minutes to the solution, stirring in an ice bath. The deep purple reaction mixture immediately turned yellow. Several attempts to recrystallize the crude product were unsuccessful. Therefore, the crude reaction was dissolved in chloroform, added 1×3 cm of silica gel to the reaction, evaporated the slurry to dryness, then added the mixture to the top of a prepared silica gel column (5×15 cm) and ran a flash column using 2:1 hexanes: ethyl acetate as eluent. The spots containing the product were combined, evaporated, then recrystallized from ethyl acetate/hexanes to afford 0.578 g (1.30 mmol, 33% yield) of the title compound as a bright orange solid: R_{f}=0.46 (2:1, hexanes: ethyl acetate); mp=113.0-114.8° C.; ¹H-NMR (CDCl₃, 200 MHz) dδ0.88 (t, 3H, J=6.8 Hz), 1.26 (br. m, 20 H), 1.57 (m, 2H), 2.61 (t, 2H, J=7.6 Hz), 6.34 (d, 1H, J =2.0 Hz), 6.84-6.91 (dd, 1 H, J=2.0 Hz, 9.8 Hz), 7.09-7.14 (dd, 1H, J=2.8 Hz, 7.3 Hz), 7.15 (s, superimposed, 1H), 7.44 (d, 1H, J=9.8 Hz), 7.81 (dd, 1H, J=0.96 Hz, 8.3 Hz); ¹³C-NMR (200 MHz, D6-DMSO) δ15.7, 24.2, 26.3, 30.5, 30.6, 30.7, 30.8, 30.9, 31.0, 31.1, 31.9, 32.0, 33.4, 106.7, 119.7, 121.8, 127.5, 131.3, 134.0, 135.0, 144.5, 148.2, 149.4, 153.5, 169.2, 185.9; MS (CI) m/z: 424 (MH+, 27); 213 (100); 185 (18); 156 (3); HRMS under EI showed no molecular ion.

Resorufin t-butylacetate (resorufin 3,3-dimethylbutyrate) 10. The procedure was the same as above but t-butylacetyl chloride (1.1 eq) was added dropwise over 10 minutes. The solution immediately turned a yellow-brown color. After 24 h, additional methylene chloride was added and then reaction was washed with saturated sodium bicarbonate, twice with distilled water, and dried over MgSO4. The resulting orange solid was triturated with ethanol and the slurry filtered to afford 0.294 g (0.945 mmol, 25% yield) of the title compound as a bright orange solid: R_{f}=0.33 (2.5:1 hexanes:ethyl acetate); mp=163.1-163.7° C.; ¹H-NMR (CDCl₃, 200 MHz) δ1.16 (s, 9H), 2.49 (s, 2H), 6.34 (d, 1H, J=1.9 Hz), 6.84-6.90 (dd, 1 H, J=1.8 Hz, 9.9 Hz), 7.09-7.14 (dd, 1H, J=2.8 Hz, 7.3 Hz), 7.15 (s, superimposed, 1H), 7.44 (d, 1H, J=10.0 Hz), 7.80 (d, 1H, J=8.3 Hz); ¹³C-NMR (200 MHz, CDCl₃) δ31.1, 32.7, 49.0, 107.7, 110.2, 119.7, 131.3, 131.4, 135.0, 135.3, 144.4, 148.2, 149.3, 153.5, 169.6, 185.8; MS (EI) m/z: 311 (M·+, 8.2); 254 (4.6); 213 (100); 185 (24); 156 (4.6); 128 (3.4); 99 (10); 57 (22); HRMS (EI): calcd. for C₁₈H₁₇NO₄: 311.1158; found: 311.1155, 0.8 ppm error.

6. Determination of Enantiomeric Purity by GC

Gas chromatography analysis was performed on a Varian 589-Series II Gas Chromatograph equipped with a Chirasil-DEX CB chiral stationary phase (25 m×0.25 mm×0.25m Chrompack, Raritan, N.J.). For analysis, solketal was converted to the acetate by dissolving the mixture of solketal and solketal butyrate in ethyl acetate (5 mL) containing acetic anhydride, 4-pyrrolidinopyridine and anhydrous potassium carbonate. The solution was stirred for one hour at room temperature, then filtered, was washed with brine, then water, dried with magnesium sulfate and evaporated to dryness. Both the starting material, solketal butyrate, and the acetate of the product were simultaneously separated with baseline resolution by using a temperature gradient (100° C. to 130° C., 2° C./min). Solketal butyrate: k′1=8.11 (S), =1.04; solketal acetate: k′1=4.21 (S), α=1.10. The ee-values reported in the tables are the mean of three injections. Racemization of the substrate during derivatization was not observed.

### EXAMPLE 2

### Hydrolase Assay

1. Hydrolase Library

The hydrolases were dissolved in BES buffer (5.0 mM, pH 7.2) at the concentrations listed in Table 1 (0.5-40 mg solid/mL solution). These concentrations can vary and the screen is valid for any concentration. CaCl₂ (2 mM) was added to the protease solutions since some proteases require calcium ions to maintain their structure. For hydrolase samples with low protein content, saturated solutions (up to 40 mg solid/ml) were utilized for hydrolase samples and with high protein content were utlized, lower concentrations (typically, 1 mg solid/ml) were utilized. Each solution was centrifuged to remove insoluble material (5 min, 2,000 rpm) and titrated to a final pH of 7.2. The protein concentrations were determined using a dye-binding assay from Bio-Rad (Mississauga, ON) with bovine serum albumin (BSA) as the standard. Solutions were stored in a 96-well assay block ‘mother plate’ equipped with aluminum sealing tape (2 mL maximum volume in each well, Corning Costar, Acton, Mass.) at −20° C. This ‘mother plate’ speeds up repeated screens using the same hydrolases and is a convenient way to store large libraries of hydrolases. Hydrolytic activity of the libraries is maintained over several months.

2. Screeening of Hydrolases with pH Indicators

The assay solutions were prepared by mixing solketal butyrate (420 μL of a 30.0 mM solution in acetonitrile), acetonitrile (470 μL), 4-nitrophenol (6,000 μL of a 0.9115 mM solution in 5.0 mM BES, pH 7.2) and BES buffer (5,110 μL of a 5.0 mM solution, pH 7.2). Hydrolase solutions (5 μL/well) were transferred from the mother plate to a 96-well microtiter plate using an 8-channel pipette. Assay solution (100 μL/well) was quickly added to each well using a 1,200 μL 8-channel pipette. The final concentrations in each well were 1.0 mM substrate, 4.65 mM BES, 0.434 mM 4-nitrophenol, 7.1% acetonitrile. The plate was quickly placed in the microplate reader, shaken for 10 s to ensure complete mixing and the decrease in absorbance at 404 nm was monitored at 25° C. as often as permitted by the microplate software, typically every 11 seconds. The starting absorbance was typically 1.2. Data were collected for one hour to ensure slow reactions and reactions with a lag time were detected. Each hydrolysis was carried out in quadruplicate and was averaged. The first 10 s of data were sometimes erratic, possibly due to dissipation of bubbles created during shaking. For this reason. the first 10 s of data was typically deleted from the calculation of the initial rate. Activities were calculated from slopes in the linear portion of the curve usually over the first two hundred seconds. The initial rates were calculated from the average dA/dt, using Eq. 2 where Δε=17,300 M⁻¹ cm⁻¹ (experimentally determined for our conditions) and 1=0.306 cm. To calculate specific activity (μmol/min/mg protein), the total amount of protein in each well was taken into account.

3. Screening of Commercial Hydrolases with pH Indicators under Interfacial Activation Conditions

The procedure was the same as above except that the BES buffer (5 mM, pH 7.2) contained Triton-X 100 (8.45 mM). Final concentration of Triton X-100 in the wells was 2.8 mM.

4. Small-scale Reactions with 1 mM (±)-solketal Butyrate

These small-scale reactions mimic the conditions in the microplate during pH indicator activity screening except that no indicator is present. Hydrolase solutions (50 μL) were added to solutions of (±)-solketal butyrate (3.50 mL of a 14.4 mM solution in acetonitrile) and BES buffer (46.45 mL of a 5.0 mM solution, pH 7.2) for a final reaction volume of 50 mL (1.0 mM substrate, 4.65 mM BES, 7% acetonitrile). After stirring at room temperature for a time estimated from the pH indicator screening, the mixture was extracted with diethyl ether (3×20 mL). These extracts, which contained both the ester substrate and the alcohol product, were combined, washed with water and dried with magnesium sulfate, filtered and evaporated to dryness.

5. Small-scale Reactions with 50 mM (±)-solketal Butyrate

Hydrolase solutions (250 μL for CRL, ROL, HLE, AOP, E013; 50 μL for cutinase) were added to solutions of (±)-solketal butyrate (352 μl of a 0.715 M solution in acetonitrile) and BES buffer (4,398 μl of a 5.0 mM solution, pH 7.2) for a final reaction volume of 5.0 mL (50 mM substrate, 4.65 mM BES, 7% acetonitrile). Reactions were worked up as above.

### EXAMPLE 3

### Identification of Enantioselective Hydrolases

The assay was used to screen for enantioselective hydrolases in 96-well microplates. Using pure enantiomers, the initial rates of hydrolysis of each enantiomer of solketal butyrate was measured separately. A library of commercial hydrolases (lipases, esterases and proteases) was screened for activity towards solketal butyrate, an important chiral building block in the synthesis of pharmaceuticals and biologically-active compounds (Jurczak, et al. Tetrahedron 1986, 42, 447-488). Many researchers have searched, without success, for a highly enantioselective hydrolase that could resolve this substrate (Vänttinen, et al. Tetrahedron: Asymmetry 1997, 8, 923-933 and references cited therein). The microorganism Comamonas testosteroni also catalyzes the enantioselective oxidation of (R)-solketal with an enantioselectivity of 49 (Geerlof, et al. Appl. Microbiol. Biotechnol. 1994, 42, 8-15). For hydrolysis in water, the highest enantioselectivity was 9 for a proteinase from Aspergillus oryza (Partali, et al. Tetrahedron: Asymmetry 1992, 3, 65-72) while for acylation of solketal in organic solvent, the highest enantioselectivity was 20-25 for a lipase from Pseudomonas species (lipase AK). Hydrolases that showed large differences in the initial rates of hydrolysis of the two enantiomers were further analyzed by traditional methods to determine enantioselectivities. Note that the ratio of separately measured initial rates of hydrolysis of the enantiomers is not the true enantioselectivity, so this screening provides only an estimated enantioselectivity. The true enantioselectivity is the ratio of the specificity constants (k_{cat}/K_{M}) for each enantiomer. By measuring initial rates of the enantiomers separately, competitive binding between the two enantiomers was eliminated. At saturating substrate conditions, the relative initial rates equal the relative k_{cat} values; at partially saturating conditions, the initial rates also depend on the K_{M} values. Thus, the ratio of separately measured initial rates ignores some or all of the effect of K_{M} on enantioselectivity. In spite of this inaccuracy, the relative initial rate provides an estimated enantioselectivity.

2. Optimizing Sensitivity of the Assay

Since most hydrolases have maximal activity near neutral pH, the exemplary assay provided herein was designed to be performed at pH 7.2. 4-nitrophenol was utilized as the pH indicator. The similarity of its pKₐ (7.15)(The Merck Index, 10th ed., Merck & Co.. Rahway, N.J., 1983, p. 950) to the pH of the reaction mixture ensures that changes in pH give a large and linear color change (The Merck Index, 10th ed., Merck & Co., Rahway, N.J., 1983, p. 950). The pKₐ of 4-nitrophenol (10 mg in 10 ml of doubly distilled water) was also identified by measuring the midpoint of the pH change as standardized base was added. The experimental result agreed with the reported value and did not change upon addition of 7% acetonitrile. The large difference in the extinction coefficients of the protonated and deprotonated forms (200 vs 18,000 M⁻¹ cm⁻¹ at 404 nm) gave good sensitivity. The extinction coefficients changed slightly upon addition of cosolvent and was determined experimentally. The high initial absorbance of 4-nitrophenoxide/4-nitrophenol limited the concentration to 0.45 mM. The 4-nitrophenol concentration in the solutions (0.45 mM or 0.006%) was well below its solubility limit, 0.08% and resulted in a starting absorbance of ˜1.2. BES (N,N-bis[2-hydroxyethyl]-2-aminoethanesulfonic acid) was utilized as a buffer because its pKₐ (7.15[]) (Beynon, et al. Buffer Solutions, The Basics, IRL Press, Oxford, 1996, p. 72) is identical to that of 4-nitrophenol.

3. Results of Screening Assay

Nine hydrolases showed estimated enantioselectivity above 4. The seven lipases and proteases favored the (R)-ester, while the two esterases favored the (S)-ester. The highest estimated enantioselectivities were found with horse liver esterase (HLE, estimated enantioselectivity=12), Rhizopus oryzae lipase (ROL, estimated enantioselectivity=11) and protease from Bacillus subtilis, variation Biotecus A. (BSP, estimated enantioselectivity=7). Previous workers identified Aspergillus oryzae protease (AOP) as an enantioselective hydrolase. This hydrolase was also among the nine enantioselective hydrolases (estimated enantioselectivity=5). The identification of HLE, ROL and BSP as enantioselective hydrolases towards solketal butyrate are new results from this screening.

TABLE 1
Activity of commercial hydrolases towards (±)-solketal butyrase and its enantiomers.

| Source of | | Prot. | Sup- | Activity | Activity | Activity | Estimated |
|---|---|---|---|---|---|---|---|
| active hydrolase^{a} | Wt.^{b} | [c] | plier | (±)^{c} | (R)^{d} | (S)^{a} | E^{e} |
| Lipases | | | | | | | |
| Aspergillus niger | 30 | 0 61 | [f] | 0.029 | 1.72 | 2.01 | 1.17(5) |
| Aspergillus oryzae | 7.1 | 4.5 | [g] | 0.039 | 0.157 | 0.0317 | 4.95(R) |
| Candida antarctica lipase A | 34 | 4.9 | [h] | 0.035 | 0.066 | 0.036 | 1.83(R) |
| Candida antarctica lipase B | 29 | 3.9 | [i] | 1.15 | 0.667 | 0.556 | 1.20(R) |
| Candida lipolytica | 35 | 0.23 | [f] | 0.114 | 0.381 | 0.340 | 1.12(R) |
| Candida rugosa | 31 | 0.35 | [j] | 2.76 | 4.25 | 1.85 | 2.30(R) |
| Candida rugosa (cylindracea) | 37 | 0.71 | [k] | 2.03 | 3.06 | 1.77 | 1.73(R) |
| Humicola sp. | 13 | 4.0 | [h] | 0.027 | 0.372 | 0.147 | 2.53(R) |
| Penicillin camembertu | 86 | 0 92 | [f] | 0.60 | 0.189 | 0.498 | 2.63(S) |
| Penicillin roqueforti | 57 | 0.74 | [f] | 0.151 | 0.8444 | 1.23 | 1.46(S) |
| Pseudomonas cepacia | 31 | 3.1 | [f] | 0.038 | 0.085 | 0.021 | 4.05(R) |
| Pseudomonas fluorescens | 5.2 | 0.73 | [g] | 0.091 | 0.572 | 0.565 | 1.02(R) |
| Rhizopus javanicus | 44 | 2.7 | [f] | 0.063 | 0.286 | 0.066 | 4.33(R) |
| Rhizopus oryzae | 53 | 4.1 | [f] | 0.040 | 0.124 | 0.011 | 11.3(R) |
| Thermus aquaticus | 1.1 | 0.29 | [g] | 0.148 | 0.665 | 0.606 | 1.10(R) |
| Esterases | | | | | | | |
| Acetylcholine escerase | 0.26 | 0.18 | [j] | 0.745 | 7.29 | 5.42 | 1.35(R) |
| Bacillus sp. | 1.1 | 0.58 | [g] | 0.112 | 0.532 | 0.180 | 2.96(R) |
| Bacillus stearothermophilus | 1.1 | 0.57 | [g] | 0.066 | 2.77 | 1.65 | 1.68(R) |
| Bacillus thermoglycosidastus | 0.82 | 0.76 | [g] | 0.065 | 1.22 | 0.470 | 2.59(R) |
| Bovine clolesterol esterase | 9.8 | 0.99 | [l] | 0.402 | 1.43 | 1.73 | 1.21(5) |
| Candida lipolytica | 3.5 | 1.4 | [g] | 0.056 | 0.244 | 0.306 | 1.25(5) |
| Cutinase | 2.1 | 1.1 | [m] | 2.38 | 10.0 | 4.40 | 2.28(R) |
| E001 | 0.40 | 0.14 | [n] | 2.08 | 11.4 | 7.65 | 1.49(R) |
| E002 | 0.38 | 0.16 | [n] | 1.27 | 1.39 | 0.721 | 1.93(R) |
| E003 | 1.0 | 0.23 | [n] | 2.91 | 4.81 | 3.64 | 1.32(R) |
| E004 | 1.0 | 0.29 | [n] | 2.21 | 3.24 | 3.40 | 1.05(S) |
| E005 | 1.0 | 0.27 | [n] | 1.88 | 1.38 | 1.10 | 1.25(R) |
| E006 | 0.64 | 0.13 | [n] | 3.59 | 12.3 | 8.80 | 1.40(R) |
| E007 | 2.1 | 0.97 | [n] | 1.26 | 1.22 | 1.35 | 1.11(S) |
| E009 | 1.0 | 0.37 | [n] | 2.65 | 4.66 | 4.01 | 1.16(R) |
| E010 | 1.0 | 0.26 | [n] | 1.56 | 4.19 | 3.01 | 1.39(R) |
| E011 | 0.80 | 0.22 | [n] | 3.44 | 4.21 | 7.30 | 1.73(5) |
| E013 | 1.0 | 0.24 | [n] | 0.869 | 0.860 | 0.843 | 1.02(R) |
| E014 | 1.0 | 0.31 | [n] | 0.136 | 0.875 | 0.498 | 1.76(R) |
| E016 | 1.0 | 0.26 | [n] | 1.22 | 1.76 | 0.960 | 1.83(R) |
| E017^{b} | 1.0 | 0.33 | [n] | 1.12 | 0.694 | 0.620 | 1.12(R) |
| E018 | 2.0 | 0.76 | [n] | 0.135 | 0.279 | 0.54 | 1.96(5) |
| E019 | 0.60 | 0.20 | [n] | 3.70 | 6.45 | 7.79 | 1.21(5) |
| E020 | 0.44 | 0.16 | [n] | 4.97 | 8.35 | 9.21 | 1.10(5) |
| Pig Liver esterase | — | 0.09 | [j] | 71.9 | 3.03 | 14.9 | 4.91(5) |
| Pig (hog) liver esterase | 0.36 | 0.49 | [g] | 5.79 | 2.26 | 6.17 | 2.73(5) |
| Horse liver esterase | 1.7 | 0.59 | [g] | 0.975 | 0.168 | 2.05 | 12.2(5) |
| Saccharomyees cervisiae | 1.2 | 0.26 | [g] | 0.085 | 0.219 | 0.074 | 2.96(R) |
| Proteases | | | | | | | |
| Aspergillus oryzae | 29 | 7.0 | [j] | 0.097 | 0.157 | 0.032 | 4.91(R) |
| Aspergillus satoi | 32 | 0.40 | [j] | 0.748 | 3.17 | 3.13 | 1.01(R) |
| Bacillus lichenformis | 5.2 | 2.1 | [g] | 0.335 | 0.261 | 0.083 | 3.14(R) |
| Bac. subtilis var. Biotecus A | 4.2 | 1.7 | [g] | 0.351 | 0.221 | 0.0303 | 7.29(R) |
| Subtilisin Carlsberg | 9.7 | 4.4 | [g] | 0.083 | 0.213 | 0.082 | 2.60(R) |
| Strepmomyces griseus | 20.1 | 7.2 | [o] | 0.012 | 0.032 | 0.0135 | 2.37IR) |
| Themsolysin, Type X | 2.4 | 0.15 | [j] | 0.095 | 0.255 | 0.738 | 2.89(5) |
| Proteinase, bacterial | 4.7 | 2.1 | [g] | 0.186 | 0.154 | 0.028 | 5.50(R) |
| Proteinase K | 0.42 | 0.07 | [g] | 2.14 | 3.08 | 2.29 | 1.24(R) |

Although only the first 3-4 minutes of data was used in the calculations, the reactions were monitored for one hour to ensure that slow hydrolases or hydrolases that show a lag time were not overlooked. All substrate/hydrolase solutions were prepared and measured in quadruplicate to ensure accuracy, although this is not necessary. The total screening time for seventy-two hydrolases in quadruplicate was 180 minutes plus several minutes between each plate to fill the 96-well plates. This time could be easily reduced to minutes with a shorter screening time. Complete screening of the library towards a racemate and its enantiomers is easily completed in an afternoon. Robotics could also be utilized to increase the speed at which these reactions are carried out.

The reaction conditions in the assay were altered in an attempt to increase the estimated enantioselectivity toward solketal butyrate. For example, the activity of hydrolases, especially lipases, often increases in the presence of an interface. It was reasoned that this interfacial activation may also change the enantioselectivity. The hydrolase library was screened with Triton X-100 (a non-ionic detergent) added to create micelles. The reaction rates increased for eight hydrolases (three lipases and five esterases), decreased for thirty-two hydrolases, and stayed constant for twelve hydrolases. Unfortunately, the estimated enantioselectivities remained unchanged or decreased slightly for the best hydrolases: AOP (decrease from 4.9 to 4.2), ROL (decrease from 11.3 to 9.6), HLE (decrease from 12.2 to 7.6), BSP (decrease from 7.3 to 5.4). With non-selective hydrolases, the estimated enantioselectivities showed small increases or decreases. For example, subtilisin Carlsberg increased from 2.6 to 2.8, esterase from Bacillus stearothermophilus increased from 1.7 to 2.4 and Aspergillus oryzae lipase decreased from 4.95 to 1.8. Overall, the selectivities towards solketal butyrate did not significantly change upon addition of Triton X-100.

To confirm these screening results, the enantioselectivity of three selective hydrolases and three poorly-selective hydrolases was determined using the conventional endpoint method, Table 2. Under conditions similar to those in the screening solutions (1 mM substrate, 7% acetonitrile as cosolvent), the true enantioselectivity and the estimated enantioselectivity agreed to within a factor of 2.3. Since 1 mM solketal butyrate is too dilute for practical preparative reactions, the enantioselectivity of these hydrolases at 50 mM solketal butyrate was also measured where the reaction mixture contained insoluble droplets of substrate. The enantioselectivity under these conditions also agreed with the enantioselectivity estimate from screening to within a factor of 2.6.

TABLE 2
True enantioselectivities of hydrolases towards solketal butyrate measured by the
endpoint method

| | S^{a} | Time^{b} | eeₐ^{c} | eeₚ^{c} | C^{d} | |
|---|---|---|---|---|---|---|
| Hydrolase | (mM) | (h) | (%) | (%) | (%) | True E^{e} |
| Rhizopus oryzae lipase | 1 | 16.5 | 95.4 | 23.6 | 80.2 | 5.0 ± 0.1 (R) |
| Rhizopus oryzae lipase | 50 | 1.25 | 37.8 | 51.2 | 42.5 | 4.4 ± 0.1 (R) |
| Horse liver esterase | 1 | 4.0 | 40.3 | 81.8 | 33.0 | 14.84 ± 0.7 (S) |
| Horse liver esterase | 50 | 2.5 | 22.1 | 77.3 | 22.2 | 9.7 ± 0.1 (S) |
| Cutinase | 1 | 2.0 | 92.8 | 27.4 | 77.2 | 5.0 ± 0.02 (R) |
| Cutinase | 50 | 0.42 | 70.3 | 41.8 | 62.7 | 4.8 ± 0.04 (R) |
| Aspergillus oryzae | 1 | 4.0 | 10.5 | 65.8 | 13.8 | 5.4 ± 0.1 (R) |
| protease | | | | | | |
| Aspergillus oryzae | 50 | 14 | 11.1 | 62.0 | 15.2 | 4.8 ± 0.1(R) |
| protease | | | | | | |
| Candida rugosa lipase | 1 | 2.5 | 86.3 | 14.4 | 85.7 | 3.0 ± 0.1 (R) |
| Candida rugosa lipase | 50 | 0.1 | 15.7 | 40.5 | 27.8 | 2.7 ± 0.1 (R) |
| Esterase E013 | 1 | 9.5 | 0 | 0 | 20 | 1.0 |
| Esterase E013 | 50 | 2.0 | 0 | nr^{g} | — | nr |

^{a}Substrate concentration in the reaction mixture.
^{b}Reaction time.
^{c}Measured enantiomeric purity of the starting material (eeₛ) or product (eeₚ).
^{d}Degree of conversion calculated by eeₛ/(eeₛ + eeₚ).
^{e}The true enantioselectivity was calculated from ees and eep according to Ref. [3]. The absolute configuration of the fast-reacting ester is in parentheses. The error limits were estimated from enantioselectivities measured from three separate GC injections.
^{f}Values from TABLE 1. [g] nr = no reaction.

The most enantioselective hydrolase was HLE, E=14.8 at 1 mM, E=9.7 at 50 mM substrate concentration, respectively. At 50 mM substrate without acetonitrile, the enantioselectivity of HLE declined slightly again to E=8.7 (c=17.1% after 2.5 h). These values agree well with the estimated enantioselectivities of 12.1 (without Triton X-100) and 7.6 (with Triton X-100). Although Partali et al. reported an enantioselectivity of 9 for AOP, we measured an estimated enantioselectivity of 4.9 and a true enantioselectivity of 4.8 under our conditions. Although the estimated enantioselectivity for ROL was also high (11.3), the true enantioselectivity was lower, E=4.8-4.9. Hydrolases with low estimated enantioselectivities (CRL, Esterase E013, cutinase) also showed low true enantioselectivities. Thus, hydrolases identified as enantioselective indeed were enantioselective and hydrolases identified as nonselective were not enantioselective. Thus, this screening procedure quickly identified HLE as a new hydrolase for the resolution of solketal butyrate with modest enantioselectivity. It is the most selective hydrolase reported in literature to date for the hydrolysis of an ester of solketal.

4. Quantitative Validation of the Assay

To confirm that color changes accurately measured the release of protons, we experimentally determined the factor Q and compared it to the theoretical Q, calculated using Eq. 1. First, proton release upon hydrolysis of the substrate was mimicked by addition of HCl (FIG. 2). Other acids such as acetic acid also show the same results. The absorbance decreased linearly due to protonation of the 4-nitrophenoxide. The reciprocal of the slopes corresponds to the buffer factor, Q, calculated using Eq. 1. The decreases were all linear and the slopes increased with decreasing buffer concentration. However, below 2 mM buffer, the experimentally measured slopes disagreed with the theoretical slopes by more than 10%. Thus, 5 mM was selected as the buffer (BES) concentration for the assay as a compromise between accuracy and sensitivity.

Small changes in reaction conditions did not compromise the sensitivity or accuracy of this assay. The measured value of Q did not change by more than the experimental error (˜5%) upon addition of 7% of acetonitrile or dimethyl sulfoxide. The measured value of Q also did not change due to unknown buffer salts in the commercial hydrolases or due to added CaCl₂ (2 mM) in the stock solutions of proteases.

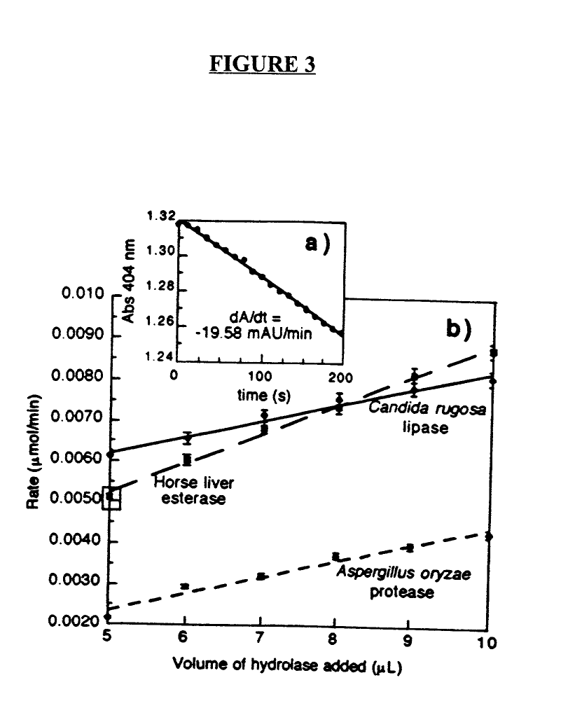

As a test reaction, horse liver esterase-catalyzed hydrolysis of racemic solketal butyrate was observed. The decrease of the indicator absorbance, FIG. 3A, was linear and corresponded to a specific activity of 1.85 μmol/min/mg protein. It was assumed that one proton is released for each ester group hydrolyzed at pH 7.2 due to the high pKₐ of solketal. Other esters (for example, 4-nitrophenyl esters) may release more than one proton per ester group hydrolyzed and should be accounted for in Eq. 1. Control experiments with no substrate or with no esterase showed no change in absorbance over one hour. When the reaction was scaled up 100-fold and monitored with a pHstat, a higher specific activity was measured (4.99 mol/min/mg protein). This difference can be attributed to activation from the rapid mechanical stirring in the pHstat experiment.

Reaction rates increased linearly with the amount of enzyme added for three typical hydrolases, one from each class of hydrolases in the library, indicating that the enzyme-catalyzed reaction rates determined with this assay are proportional to the total enzyme concentration, FIG. 3B.

### EXAMPLE 5

### Quick E Assay

The quick E method is an extension of the pH-indicator assay described in the previous example provides several advantages over presently available assays. As shown herein, the quick E method provides rapid and accurate results, requires small amounts of hydrolase, and is a simpler assay than those currently in use. This assay was performed as indicated below.

1. Quick E Measurements of Solketal Butryate

Hydrolysis of an ester bond releases an alcohol and acid moiety and also a proton(s) as shown above. With a suitable pH-indicator, the released proton(s) produces a change in absorbance of the pH indicator. If the conditions of the assay are chosen carefully, the rate of change of absorbance of the pH-indicator is directly proportional to the rate of enzyme-catalyzed ester hydrolysis, Eq. 1, where 404 nm is the difference in extinction coefficients of the fully protonated and deprotonated form of the pH indicator. Importantly, the reaction buffer and pH indicator must have identical pKₐ values to ensure a linear relationship between the absorbance kinetics and the enzyme kinetics. This sensitive pH indicator assay allows for accurate measurement of the rates of hydrolysis of the pure enantiomers of siketal butyrate separately, Eq. 1.

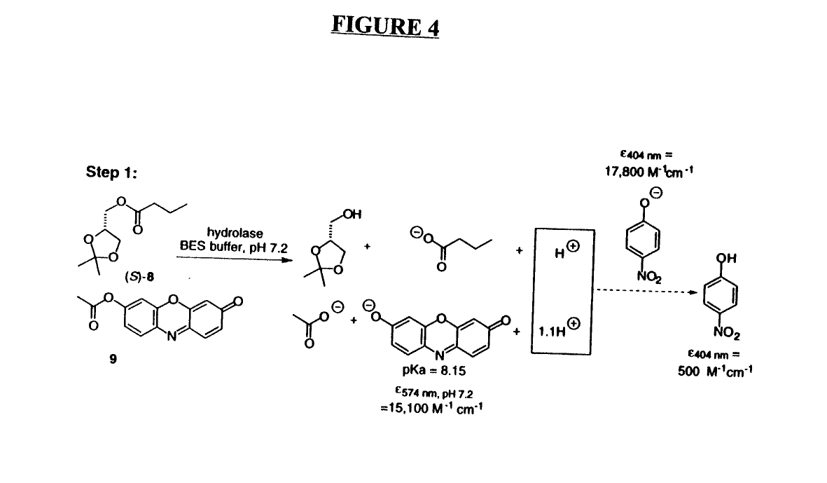

To introduce competition into the separately-measured initial rate measurements of the pure enantiomers of solketal butyrate, a reference compound such as resofurin acetate can be added, followed by measurement of their relative rates of hydrolysis, FIG. 4.

The rate of hydrolysis of the reference compound is easily calculated if the extinction coefficient of resorufin for the screening conditions is determined, Eq. 6. Under the screening conditions shown herein, ε=15,100 M−1cm−1 for pH 7.2, 7% acetonitrile was measured.

$${Rate}_{reference} ( µmol \text{/} min ) = \frac{ⅆ {A}_{404 nm}}{ⅆ t} \times \frac{1}{Δ {ϵ}_{574 nm , pH 72} \times 1} \times r x n vol . \times {10}^{6} \qquad ( Eq . 6 )$$

To determine the rate of hydrolysis of the pure enantiomer of solketal butyrate in the presence of the reference compound, the acid dissociation constant of resorufin alcohol (pKₐ=8.15) was first determined. Only 10% of the resorufin released upon hydrolysis of resorufin acetate is deprotonated at pH 7.2, so 1.1 protons are released for every resorufin acetate molecule hydrolyzed (one proton is from the acetic acid which is fully deprotonated at neutral pH). Since the pH indicator detects all protons, the protons from hydrolysis of the reference compound from the total rate of protons detected was determined to obtain the true rate of enzyme-catalyzed hydrolysis of the pure enantiomers of solketal butyrate, Eq. 7.

$$initial {rate}_{S - substrate} ( µmol \text{/} min ) = ( \frac{ⅆ {A}_{404 nm}}{ⅆ t} \times \frac{[ Buffer ]}{[ indicator ]} \times \frac{1}{Δ {ϵ }_{404 nm} \times 1} \times r x n vol . \times {10}^{6} ) - 1.1 ( {rate}_{reference} ) \qquad ( Eq . 7 )$$

Note that for Eq. 7 to be accurate, it is important to use a strong enough buffer during the assay to ensure that the pH of the solution overall does not change significantly during quick E measurements. The ratio of deprotonated resorufin molecules changes with changing pH. 5 mM BES buffer as a compromise between high buffer concentrations to ensure small changes in pH throughout the assay (0.05 pH units for 10% hydrolysis of the substrates) and low buffer concentrations to maximize the pH indicator sensitivity (<0.055 change in absorbance units for 10% hydrolysis of substrates in a quick E measurement).

The ratio of initial rates of hydrolysis of a pure enantiomer of solketal butyrate versus a reference compound yields the selectivity ratio after taking into account the initial concentrations of the substrate, Eq. 8.

$$\frac{( S ) - 1}{reference} selectivity = \frac{{( {K}_{cat} / {K}_{M} )}_{( S ) - 1}}{{( {K}_{cat} / {K}_{M} )}_{reference}} = \frac{{v}_{( S ) - 1} \times [ reference ]}{{v}_{reference} \times [ ( S ) - 1 ]} \qquad ( Eq . 8 )$$

The experiment is repeated with the opposite enantiomer and the ratio of selectivity ratios yields the true enantioselectivity, Eq. 9.

$$E = \frac{( S ) - 1}{( R ) - 1} selectivity = \frac{\frac{( S ) - 1}{reference} selectivity}{\frac{( R ) - 1}{reference} selectivity} \qquad ( Eq . 9 )$$

The extended quick E method was utilized to measure the quick E values of several hydrolases towards solketal butyrate, a common chiral building block in organic synthesis. These values were then compared to the E values determined by the endpoint method to confirm that quick E measured the enantioselectivities correctly. Enantioselectivities by quick E agrees with those by the endpoint method for five out of six hydrolases, Table 3. Only with horse liver esterase was a two-fold difference between the endpoint E and the quick E value observed. This can be attributed to the lower quick E due to the added surfactant, Triton X-100, which was not present during the endpoint reaction.

TABLE 3
Enantioselectivities of hydrolases towards (±)-solketal butyrate, 8 using the
Quick E method.

| | Time | (S)-enantiomer + ref. | | (R)-enantiomer + ref^{c} | | | |
|---|---|---|---|---|---|---|---|
| Hydrolase^{a} | (s)^{b} | 404 nm | 575 nm | 404 nm | 574 nm | Quick E^{d} | Endpoint E^{e} |
| Aspergillus oryzae Protease | 10-100 | 0.00696 | 0.0998 | 0.0455 | 0.103 | 6.3 ± 1 8 (R) | 5.4 ± 0.1 (R) |
| Candida rugosa lipase | 0-100 | 0.208 | 0.855 | 0.731 | 0.800 | 3.7 ± 0.8 (R) | 3.0 ± 0.1 (R) |
| Cutinase from Fusarium solani | 0-200 | 1.07 | 4.57 | 4.06 | 5.82 | 3.0 ± 1.1 (R) | 5.0 ± 0.02 (R) |
| Esterase E013 | 0-100 | 0.267 | 4.38 | 1.04 | 11.7 | 1.4 + 0.3 (R) | 1.02 |
| Horse liver esterase | 0-200 | 2.55 | 0.188 | 0.225 | 0.137 | 8.3 ± 2.1 (S) | 14.8 ± 0.7 (S) |
| Rhizopus oryzae lipase^{f} | 0-100 | 0.970 | 0.922 | 0.268 | 0.453 | 5.6 ± 1.4 (R) | 5.0 ± 0.1 (R) |
| ^{a}See TABLE 5. | | | | | | | |
| ^{b}Data collection time. | | | | | | | |
| ^{c}Final concentration in the microplate wells during assay: 0.10 mM resorufin acetate, 1.0 mM (R)- or (S)- solketal butyrate, 7% acetonitrile, 0%. 134 mM (0.86%) Triton X-100, 0.434 mM 4-nitrophenol, 4.65 mM BES buffer, pH 7.2. Changes in absorbance at 404 nm and 574 nm were monitored simultaneously. Rates are calculated in mol/min/mg protein using equations 5.10 and 5.11 and taking into account the amounts of protein in each well as determined by the | | | | | | | |
| # Bio-Rad assay using BSA as a standard. No spontaneous chemical hydrolysis was detected. | | | | | | | |

^{d}Enantiomeric ratio calculated using Equation 6.7. Reported error is the standard deviation for 3 measurements. Endpoint values were determined using the identical conditions as in the microplate wells during screening but no pH indicator or Triton X-100 was added: Janes, et al., Eur. J. 1998, 4, 2317-2324. Resorufin t-butylacetate 10 was used as a reference compound in the quick E measurement at 0.1 mM concentration in the well during assay.

2. Quick E Measurements of Solketal Butyrate 8 Using pH Indicators

These measurements were carried out using 96-well microplates and a microplate reader. The assay solutions were prepared by mixing the pH-indicator, 4-nitrophenol (3,000 L of a 0.9115 mM solution in 5.9 mM BES, pH 7.2), BES buffer (2560 μL of a 5.0 mM solution containing 0.33 mM (2.11%) Triton X-100, pH 7.2), and acetonitrile (28.6 μL), then vortexing the solution. (R)-solketal butyrate (R)-8 (37.4 μL of a 168.6 mM solution in acetonitrile), and resorufin acetate, 9 (374 μL of a 1.685 mnM solution in acetonitrile) were added dropwise to the slowly vortexing solution to ensure the formation of micelles and clear solutions. Hydrolase solutions (5 μL/well) were added to the wells and the assay solution (100 μL/well) was added quickly using an 8-channel pipette. The final concentration in each well is 1.0 mM solketal butyrate, 0.1 mM resorufin acetate, 4.65 mM BES buffer, 0.434 mM 4-nitrophenol, 0.134 mM (0.86%) Triton X-100, 7% acetonitrile. The plate was placed quickly in the microplate reader, shaken for 10 s to ensure complete mixing and the simultaneous decrease in absorbance at 404 nm and increase at 574 nm were monitored at 25° C. as often as permitted by the microplate software, typically every 11 seconds. Data were collected for 15 minutes. Each hydrolysis was carried out in quadruplicate and was averaged. The procedure was repeated for the other enantiomer (S)-8. Activities were calculated with equations 5.10 and 5.11 usinng the slopes of the 15,140 M−1 cm−1 (both experimentally determined for our conditions) and 1=0.306 cm. To calculate the specific activities (μmol/min/mg protein), the total amount of protein in each well as determined by the Bio-Rad protein Assay was taken into account.

3. Quick E With Chromogenic Substrates

The Quick E method can also be carried out without pH indicators if a chromophore is released upon hydrolysis of the ester directly, such as 4-nitrophenyl esters are carboxylic acids. To properly evaluate enantioselectivity based upon initial rates of hydrolysis, the rates of hydrolysis of both enantiomers must be measured simultaneously to reflect their relative binding in the enzyme's active site. However, the relative rate of hydrolysis of both enantiomers cannot be simultaneously determined without measuring enantiomeric purities, so researchers often estimate enantioselectivity by measuring the initial rates of hydrolysis of the two enantiomers in separate hydrolysis experiments, Eq. 10.

$$Estimated E ≅ \frac{initial {rate}_{fast enantiomer}}{initial {rate}_{slow enantiomer}} \qquad ( Eq . 10 )$$

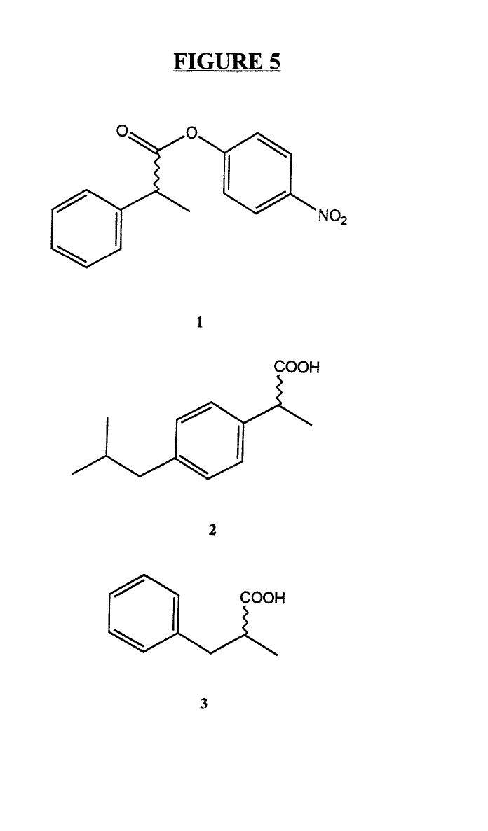

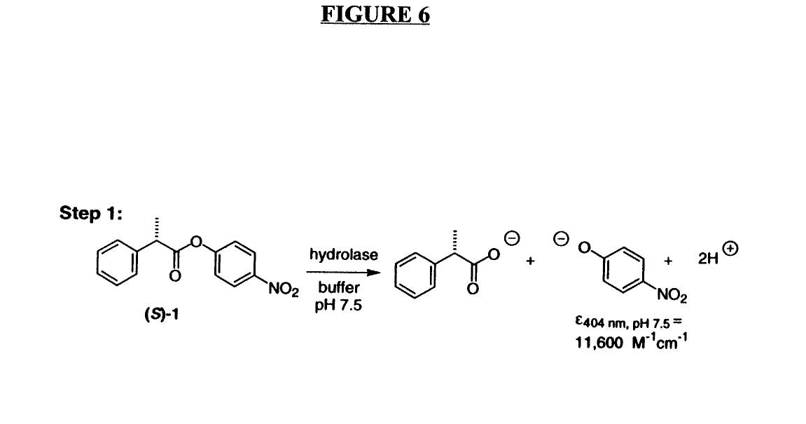

The initial rates of hydrolysis of 4-nitrophenyl esters of pure enantiomers were determined to quickly estimate the enantioselectivity of several lipases towards three common chiral carboxylic acids: 2-phenylpropanoic acid, p-nitro phenyl ester (or, 4-nitrophenyl 2-phenylpropanoate), 1; 2-(4-isobutylphenyl)propanoic acid (commercially available as Ibuprofen), 2; and phenylalanine, 3, FIG. 5. The rates of hydrolysis of 4-nitrophenyl esters were conveniently determined spectrophotometrically by monitoring the rate of release of 4-nitrophenoxide anion at 404 nm, FIG. 6. The ratio of separately measured initial rates of hydrolysis estimated the enantioselectivity. The quick E of phenylalanine, 3, was not measured because of its instability in aqueous solution.

The ratio of rates over- or underestimated E by as much as 70 percent because it ignored competitive binding of the two enantiomers to the enzyme. In other cases, researchers found that differences in K_{M} for the enantiomers contributed a factor of three to four to the enantioselectivity (Wu, et al. J. Am. Chem. Soc. 1990, 112, 1990-1995; van der Lugt, et al. in Microbial Reagents in Organic Synthesis, Servi, S., Ed. Kluwer Academic, 1992, pp 261-272).

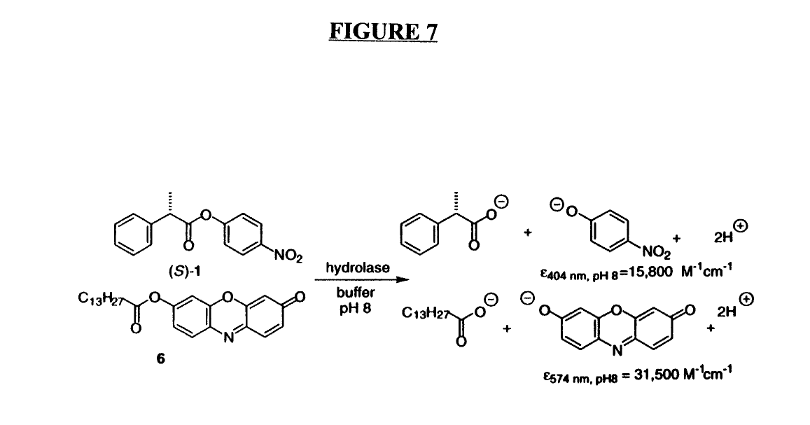

To reintroduce competition, resorufin tetradecanoate was added as a reference compound, FIG. 7. The initial rates of hydrolysis of (S)-1 were monitored at 404 nm and the reference compound at 572 nm in the same solution. After taking into account the initial concentrations of both substrates, the ratio of these rates yielded the selectivity of the hydrolase for (S)-1 over the reference compound, Eq 8.

A second experiment using (R)-1 and the reference compound yielded the selectivity of (R)-1 over the reference compound. The ratio of these two selectivities yields the enantiomeric ratio, Eq. 9.

The quick E method agreed with the endpoint method for both 1 and 2 using five different lipases (Table 3). Low (E=1.4), average (E=27) and excellent (E=210) enantiomeric ratios were measured correctly by this technique (Table 4). Each hydrolysis experiment requires 30 seconds; thus, the measurement time for E was only one minute.

TABLE 4
Enantioselectivities of hydrolases
towards 4-nitrophenyl-(±)-2-(4-isobutylphenyl)propanoate 2 determined by the quick E method.

| | | (S)-enantiomer + ref^{b} | | (R)-enantiomer + ref^{b} | | Quick | |
|---|---|---|---|---|---|---|---|
| Substrate | Lipase^{a} | 404 nm | 574 nm | 404 nm | 574 nm | E^{c} | Endpoint E^{d} |
| 1 | Candida antarctica. type A | 0.0309 | 0.0111 | 0.0119 | 0.0107 | 2.3 ± 0.2 | 1.9 ± 0.1 |
| 1 | Candada rugosa | 0.0948 | 0.445 | 0.0260 | 0.423 | 3.5 ± 0.3 | 3.5 ± 0.2 |
| 1 | IPA-Candida rugosa | 0.102 | 0.206 | 0.000215 | 0.100 | 230 ± 20 | >100 |
| 1 | Pseudomonas cepacia | 0.170 | 0.0636 | 0.00488 | 0.0487 | 27 ± 3 | 29 ± 3 |
| 1 | Porcine pancreas | 0.00105 | 0.000495 | 0.000659 | 0.00437 | 1.4 ± 0.2 | 1.1 ± 0.1 |
| 2 | Candida rugosa | 0.0677 | 0.494 | 0.0266 | 0.523 | 2.7 ± 2 | 1.2 ± 0.1 |
| 2 | IPA-candida rugosa | 0.116 | 0.239 | <0.0005 | 0.157 | >140 | 55 ± 5 |
| 2 | Pseudomonas cepacia | 0.00430 | 0.0441 | 0.00740 | 0.0306 | 2.5 ± 0.3 (R) | 1.1 ± 0.1 (R) |

^{a}See TABLE 3 for details.
^{b}Final concentration in the cuvette during assay: 0.071 mM resorufin tetradecanoate, 0.351 mM (R)- or (S)-ester, 9% acetonitrile, 0.73% X-100, 43 mM Tris buffer, pH 8. The liberated 4-nitrophenxide and resorufin were measured at 404 nm and 574 nm, respectively, at 25° for 15 seconds. No measurable spontaneous hydrolysis was detected. Rates are calculated in μmol/min/mg protein using l + 1 cm, ε₅₇₄ₙₘ = 31,500 M⁻¹ cm⁻¹.
^{c}Enantiomeric ratio calculated using the equation ((initial rate_{(S)-1}/initial rate₆) × (initial rate₆/initial rate_{(R)-1})). Error is the standard deviation for 3 measurements.
^{d}Values from TABLE 5
^{e}CRL solution (214 mg/mL; 1.9 mg protein/mL).

TABLE 5
Enantiomeric ratios of hydrolases towards 4-nitrophenyl-esters 1-2,
using the endpoint method^{a} and Quick E

| Sub- | | Wt^{a} | Sup- | | | | | E^{b} | Initial Rate | Initial Rate | Estimated |
|---|---|---|---|---|---|---|---|---|---|---|---|
| strate | Lipase^{b} | (prot.) | plier | eeₛ^{c} | eeₚ^{e} | Time^{f} | C | endpoint | (S)ⁱ | (R)ⁱ | E^{j} |
| 1 | Candida antarctica | 18 2 | ^{k} | 20 | 23 | 6.1 | 37 | 1.9 ± 0.1 | 0.0203 | 0.00515 | 4.0 ± 0 2(S) |
| | type A | (2.62) | | | | | | | | | |
| 1 | Candida rugosa | 36.9 | ^{l} | 17 | 50 | 20.3 | 25 | 3.5 ± 0.2 | 0.263 | 0.223 | 1.2 ± 0.2(S) |
| | | (0.406) | | | | | | | | | |
| 1 | IPA-Candida rugosa | Na | ^{m} | 43 | 98 | 4.3 | 30 | 100 | 0.193 | 0.00500 | 40 ± 2(S) |
| | | (0.70) | | | | | | | | | |
| 1 | Pseudumonas cepacia | 29.3 | ^{k} | nd | 67 | 2.4 | 60 | 29 ± 3 | 0.198 | 0.0100 | 20 ± 1 (S) |
| | | 2.93 | | | | | | | | | |
| 1 | Procine pancreas | 42.3 | ^{l} | nd | 2.0 | 22.0 | 4.0 | 1.1 ± 0.1 | 0.00590 | 0.00403 | 1.4 ± 0.1 (S) |
| | | (7.15) | | | | | | | | | |
| 2 | Candida rugosa | 18.2 | ^{l} | 5.3 | 14.5 | 4.25 | 27 | 1.4 ± 0.1 | 0.00478 | 0.00397 | 1.2 ± 0.1 (S) |
| | | (2.62) | | | | | | | | | |
| 2 | IPA-Candida rugosa | NA | ^{m} | 40.1 | >99 | 21.0 | 29 | >100 | 0.0382 | 0.000695 | 55 ± 5 (S) |
| | | (0.70) | | | | | | | | | |
| 2 | Pseudomonas cepacia | 29.3 | ^{k} | 28 | 43.2 | 16.5 | 39 | 3.3 ± 0.1 | 0.00694 | 0.0075 | 1.1 ± 0.1 (R) |
| | | (2.93) | | | | | | (R) | | | |
| 3 | Aspergillus niger | 49.3 | ^{k} | 0 | 0 | 0.3 | 100^{a} | nd | 0.357 | 0.266 | 1.34 ± 1 (S) |
| | | (1.38) | | | | | | | | | |
| ^{a}Reaction conditions: room temperature, 9 mL 10 mM Tris buffer, pH 8.0 100 mgsolid lipase powder or 0.7 mg protein of IPA-CRL, 100 mg substrate dissolved in 1 mL acetonitrile except for 3:8 mL 10 mM sodium acetate buffer, pH 5, 208 mg solid lipase, 100 mg substrate dissolved in 1 mL acetonitrile. | | | | | | | | | | | |
| ^{b}For spectrophotometric measurements, the lipases were dissolved in Tris buffer (25 mM, pH 7.5) and centrifuged to remove insoluble material, the following lipases showed no activity (<1 × 10⁻⁵ μmol/min/mL) towards either enantiomer of 4-nitrophenol-2-phenylpropanoate over 60 seconds: Aspergillus niger AP-6 (Amano, 45.0 mg/mL), Candida lypolytica (Amano, 39.0 mg/mL), Chromobacterium viscosum (Sigma-Aldrich, 6.5 mg/mL), | | | | | | | | | | | |
| # Mucor javanicus (Amano, 40.2 mg/mL), Rhizopus oryzae (Amano, 30.9 mg/mL), Rhizopus stolonifer (Amano, 32.0 mg/mL), wheat germ (Sigma-Adrich, 16.1 mg/mL). | | | | | | | | | | | |

^{c}Amount (mg) of solid enzyme per mL of buffer in the stock solutions used for initial rate measurements.
“na” = not available
^{d}Protein concentration of stock solutions in mg/mL determined by the Bio-Rad assay using BSA as the standard.
^{e}Enantiomeric ratio calculated from eeₓ and eeₚ as defined by Chen, C.S.; Fujimoto, Y.; Girdaukas, G.; Sih, C.J. J. Am Chem., Soc. 1982, 104, 7294-7299. All lipases preferred the (S)-enantiomer except in one example. Error limits for E were estimated assuming a 1% error in enantiomeric purity.
ⁱReaction conditions during spectrophotometric assay: 0.37 mM substrate, 0.38% Triton X-100, 45.2 mM Tris Buffer, 4.76% acetonitrile except for reaction with ANL: 0.25 mM substrate, 0.5% acetonitrile, 10 mM HEPES buffer, pH 8.0 Rates of hydrolysis are reported in μmol/min/mg protein and are calculated using the Beer-Lambert Law with ε + 15,800 M⁻¹ cm⁻¹, l + 1 cm.
^{j}Error limits are the average of 3 separate experiments.
^{k}Amano Enzyme USA Co., Ltd. (Troy, VA).
^{l}Sigma-Aldrich (Oakville, ON).
^{m}CRL partially-purified with isopropanol as outline in the experimental section.
ⁿ(±)-Phenylalanine 4-nitrophenyl ester TFA salt hydrolyzed spontaneously at pH 5 at a rate of 3.78 × 10⁻⁴s⁻¹ (T_{½}= 0.51 h). It was impossible to run the reaction and determine enantiomeric purities without complete hydrolysis of the substrate.
^{o}Rate of hydrolysis has been corrected for background chemical hydrolysis.

Using quick E, however, it is possible to measure an E of 210±20 for the IPA-CRL catalyzed hydrolysis of 1, and set a lower limit of >140 for 2, Table 3. Quick E measures these high values by measuring the rate of hydrolysis of the slow enantiomer. The inability to measure a higher enantiomeric ratio for 2 was due to the unusually low reactivity of 2. IPA-CRL favored the reference compound by a factor of 23 over the fast enantiomer of 2 and by >3000 over the slow enantiomer.

Using a slower-reacting reference compound, it may be possible to measure higher enantiomeric ratios. Another limit to measuring high enantiomeric ratios with quick E is the enantiomeric purity of the starting slow enantiomer. Reaction of the to contaminating fast enantiomer will give a measured E that is lower than the true E. If the enantiomeric purity of the slow enantiomer is >99.9% ee, then quick E should be able to measure enantioselectivities up to 1000.

The ability to measure high enantioselectivities with quick E is important because this is the goal of most screening studies. Using the endpoint method, measurement errors in conversion or enantiomeric purity limit make it difficult to measure enantiomeric ratios >100.

### EXAMPLE 6

### Use Quick E Assay For Enantioselectivity Towards Chiral Alcohols and Carboxylic Acids

1. Spectrophotometric Assays

Separately measured initial rates of chromogenic esters. 4-nitrophenyl esters of 2- phenylpropanoic acid, 1, and 2-(4-isobutylphenyl)propanoic acid, 2, were emulsified in aqueous solutions according to Vorderwuibecke. A solution of (R) or (S)-4-nitrophenyl-esster (500 μL of a 7.8 mM solution in acteonitrile) was added dropwise to Tris buffer (9000 μL, 50 mM, pH 7.5) containing 0.45 w/v % Triton X-100 (Pierce Surfact-Amps) and vortexed until clear. This emulsion remained clear for at least 3 hours. To measure the initial rates of hydrolysis, lipase solutions from Table 3 (100 μL) were added to the substrate emulsion (900 μL) in a cuvette at 25° C. and the linear increase in absorbance at 404 nm was monitored for 15 s. The final concentrations in the cuvettes were 0.369 mM substrate, 0.39% Triton X-100, 45 mM Tris buffer, 4.76% acetonitrile. No spontaneous chemical hydrolysis was detected. Solutions of 4-nitrophenyl esters of phenylalanine, 3, in acetonitrile (5 IL of a 50 mM solution in acetonitrile) were added to HEPES buffer (1000 μL, 10 mM , pH 7.5). To measure the rates of hydrolysis, 5 μL of ANL solution was added and the change in absorbance monitored as above. The reported rates are corrected for spontaneous chemical hydrolysis (t½=0.51 h).

2. Quick E Measurements of Chromogenic Esters, 1-2

Substrate solutions were the same as for separately measured initial rate measurements, but a solution of resorufin tetradecanaote 6 (0.5 mL of a 1.59 mM solution in acetonitrile) was added slowly to the vortexing solutions of pure enantiomers. It was necessary to use a 2-fold increase in the concentration of Triton X-100 to solubilize resorufin tetradecanoate. The emulsions remained clear for at least 2 hours. To measure the initial rates of hydrolysis, lipase solutions from Table 4 (100 μL) was added to the substrate emulsion (900 μL) in a cuvette at 25° C. and the linear increase in absorbances at 404 nm and 574 nm were monitored for 15 s. Rates are calculated in μmol/min/mg protein after taking into account the amount of protein in each cuvette. The final concentrations in the cuvettes were 0.351 mM substrate, 0.071 mM resorufin tetradecanoate, 0.73% Triton X-100, 43 mM Tris buffer, 9% acetonitrile. No spontaneous chemical hydrolysis was detected at 404 nm or 574 nm.

3. General Procedure for Small-scale Enzyme-catalyzed Hydrolyses of Esters to Determine the True E

Lipases (100 mg solid or 0.7 mg protein of IPA-CRL) were added to Tris HCl buffer (9 mL, 10 mM, pH 8) and stirred for 30 minutes to ensure complete dissolution. Substrates (100 mg) were dissolved in 1 rnL acetonitrile and added to the stirring solution and the rate of hydrolysis was monitored by pH stat which maintained the pH at 8 by automatic titration with 0.0965 N NaOH. Reactions were terminated by extracting the remaining starting material with diethyl ether (3×20 mL). The aqueous phase was then acidified to pH 2 with 1 N HCl and the product acid extracted with diethyl ether (3×20 mL). Ethanol was added dropwise during the workup to break up the emulsions and inactivate the enzymes. Both extracts were dried with MgSO4, filtered and concentrated in vacuo. For analysis, the remaining starting material was converted to the acid using aqueous NaOH (1.5 equiv.) in ethanol. The enantiomeric excesses were measured by HPLC as described below.

4. Enantiomeric Excess Determination by HPLC

Enantiomers of 1 and 2 were analyzed using a Daicel OD-H column at 25° C., 254 nm, 1.0 mL/min. Absolute configurations were confirmed with authentic samples. 2-phenylpropanoic acid: 98:2:1 hexanes:isopropanol:TFA; kR′=3.35; kS′=4.05; α=1.2; Rs=2.13.4-nitrophenyl-2-(4isobutylphenyl)propanoate: 100:1:0.1 hexanes:isopropanol:TFA; kR′=2.41; kS′=2.93; α=1.21; Rs=1.66.2-(4- isobutylphenyl)propanoic acid: 100:1:0.1 hexanes:isopropanol:TFA; kR′=3.31; kS′=4.27; α=1.29; Rs =2.43. Enantiomers of phenylalanine were analyzed using a Crownpak CR(+) column at 25° C., 200 nm, 0.8 mL/min, with aq. HClO₄, pH 2 as the mobile phase.Phenylalanine: kD′=3.93; kL′=5.20; α=1.32; Rs=3.86.

### EXAMPLE 7

### Quick E Screening for Enantioselective Esterases

To identify potential synthetic applications of esterases from thermophilic microorganisms, substrate selectivity and enantioselectivity toward a library of approximately fifty esters. Screening was accomplished using the Quick E method, which uses pH indicators to detect hydrolysis and a chromogenic competitive ester to establish precise selectivities. This screening showed that certain esterases obtained from _{ThermoGen}, Inc. (Chicago, Ill.) usually favor a hexanoyl acyl group, often with selectivity >1000 over an acetyl group. However, one esterase, E018b, favors acetyl over hexanoyl by a factor of seventy-six. This selectivity permits selective removal of acetyl or hexanoyl protective group. Thirteen of the nineteen esterases showed high enantioselectivity (>50) toward 1-phenethyl butyrate favoring the (R)-enantiomer. Three esterases (E008, E011, E014) show moderate or better enantioselectivity (>20) toward methyl 2-chloropropanoate favoring the (S)-enantiomer and one esterase (E004) shows high (>50) selectivity toward menthyl acetate favoring the (S)-enantiomer. This rapid identification of potential synthetic applications shows the usefulness of the Quick E method in the characterization of new enzymes.

1. Initial Screening of Hydrolases

Each well of a 96-well polystyrene microplate was filled with ester solution (7 μL of a 14.3 mM solution in acetonitrile), 4-nitrophenol solution (23 μL of a 1.935 mM in BES buffer (1 mM, pH 7.2) and BES buffer (65 μL, 1 mM, pH 7.2). The final concentrations in each well was 1 mM substrate, 0.45 mM 4-nitrophenol, 1 mM BES and 7 vol % acetonitrile. An aliqout of esterase solution (5 μL in BES buffer, (5 mM), and the decrease in absorbance at 404 nm was monitored for 20 min at 25° C. Blanks contained no substrate. When needed, the esterase concentration was increased or decreased.

2. Substrate Selectivity and Enantioselectivity Measurements

A stock solution was prepared by mixing 4-nitrophenol solution (1.293 μL of a 1.825 mM solution in 1 mM BES containing 0.33 mM Triton-X, pH 7.2), BES buffer (3.339 μL of a 1 mM solution containing 0.33 mM Triton-X, pH 7.2) and acetonitrile (65.5 (L). Substrate solution (for example, 35.1 (L of a 149 mM vinyl pivalate in acetonitrile) and resorufin ester solution (for example, 263 (L of 2 mM resorufin t-butyl acetate in acetonitrile) were added dropwise with continuous stirring to form a clear emulusion stable for at least several hours. This solution was pipetted into a 96-well polystyrene microplate (100 μL/well). Esterase solution (5 μL in 5 mM BES) was added to each well and the microplate was placed in the microplate reader, shaken for 5 s and the decrease in absorbance at 404 nm and the increase in absorbance at 574 nm were measured as often as permitted by the microplate reader, typically every 11 s. Data was collected for 20 minutes, at 25° C., in triplicate and was averaged. Activities were calculated from the equations above using the linear, initial portions of the curve. Final concentrations in the well were 1.0 mM substrate (vinyl pivalate), 0.10 mM resorufin t-butyl acetate, 0.45 mM pNP, 1.12 mM BES, 0.29 mM Triton-X, 7% AcCN.

Similar types of calculations have previously been done using other methods of recording the selectivities, most notably fluorescent labeling followed by Edman degradation of peptide sequences (Petithory, et al. Proc. Natl. Acad. Sci. USA, 1991, 88, 11510-11514) and HPLC coupled with mass spectrometry (Birkett. et al. Anal. Biochem. 1991, 196, 137-143; Berman, et al. J. Biol. Chem. 1992, 267, 1434-1437). Additionally, the derivation of equations for treatment of these situations was done by Schellenberger (Schellenberger, et al. Biochemistry 1993, 32, 4344-4348).

3. Screening Method

Initial screening measures the initial rate of hydrolysis of different esters. The rate of reaction was measured by monitoring the release of protons using a pH indicator, 4-nitrophenol. In this assay, the yellow color of the phenoxide disappears as the reaction proceeds. The amount of ester hydrolyzed can be measured quantitatively, as is known in the art.

Although comparing the separately measured initial rates of two substrates gives an idea of which substrate the hydrolase prefers, it does not give a quantitative measure of the selectivity of the hydrolase. The selectivity of an enzyme is determined by both the relative k_{cat} (rate of reaction) and relative K_{M} (approximately, the binding of the substrate). For example, imagine two substrates that react at the same rate, but one binds better. In a competitive experiment, the better binding one will selectively react, but if the rates are measured separately, both will react at similar rates (assuming the concentration of the substrate is above K_{M}). For this reason, the ratio of separately measured initial rates does not measure the true selectivity of the enzyme. A quantitative measure requires a competitive experiment; that is, allowing both substrates to compete for the active site. One way to measure selectivity is to allow the two substrates to compete against each other. Indeed, researchers previously measured the chain length selectivity of hydrolases by mixing substrates and measuring the relative amounts of hydrolysis TLC or GC (Berger, et al. Biotechnol. Lett. 1991, 13, 641-645; Sugiura, et al. Chem. Pharm. Bull. 1975, 23, 1226-1230).

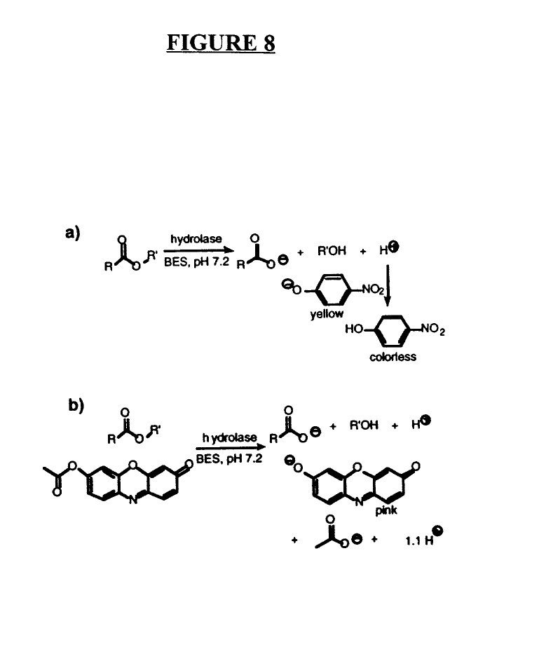

However, it is faster to measure selectivity of each substrate as it competes against a third substrate, which we call a reference compound. The ratio of the two competitive reactions is the true selectivity. As a reference compound, resorufin acetate, which yields the pink resorufin upon hydrolysis, was used as shown in FIG. 8. Accurate measure of the selectivities requires that the substrate and the reference compound react at comparable rates. Since some substrates react rapidly while others react slowly, we require both fast-reacting and slow-reacting reference compounds. In some cases we replaced resorufin acetate with the slower reacting resorufin pivaloate or resorufin isobutyrate as reference compounds.

4. Determination of Substrate Selectivity

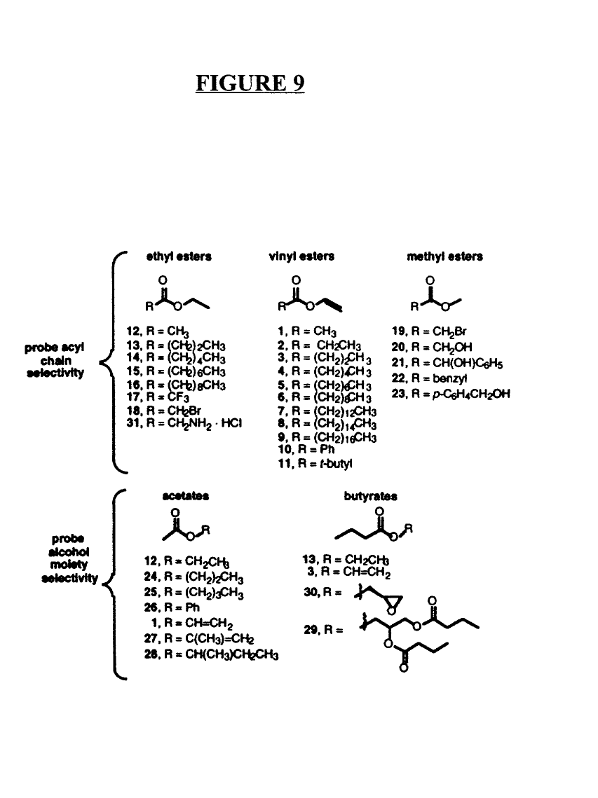

To survey the substrate selectivity of the _{ThermoGen} esterases, the ability of the enzymes to hydrolyze thirty-one different commercially-available esters, Scheme 1 (FIG. 9, Table 6).

TABLE 6
Specific activities of ThermoGen esterases toward esters 1-31.^{a}

| | Esterase | | | | | | | | | | | | | | | | | | |
|---|---|---|---|---|---|---|---|---|---|---|---|---|---|---|---|---|---|---|---|
| Substrate | E001 | E002 | E003 | E004 | E005 | E006 | E007 | E008 | E009 | E010 | E011 | E012 | E013 | E014 | E015 | E016 | E018^{b} | E019 | E020 |
| 1 | 150 | 160 | 63 | 60 | 140 | 120 | 2.3 | 12.0 | 340 | 5.5 | 5.1 | 3.1 | 3.0 | 1.2 | 89 | 63 | 150 | 120 | 110 |
| 2 | 940 | 730 | 220 | 210 | 780 | 260 | 130 | 270 | 1400 | 140 | 160 | 11 | 140 | 170 | 350 | 220 | 20 | 400 | 370 |
| 3 | 860 | 1000 | 300 | 790 | 550 | 410 | 76 | 320 | 2000 | 190 | 220 | 37 | 240 | 180 | 280 | 230 | 1.1 | 490 | 550 |
| 4 | 4900 | 3000 | 1400 | 760 | 4100 | 1700 | 510 | 980 | 3800 | 630 | 680 | 48 | 660 | 540 | 1100 | 1300 | 9.3 | 2400 | 3000 |
| 5 | 2100 | 1700 | 1600 | 960 | 1200 | 960 | 160 | 300 | 1800 | 220 | 260 | 50 | 190 | 8.0 | 320 | 1200 | 2.0 | 880 | 1200 |
| 6 | 1800 | 1600 | <5 | 150 | 1500 | 0.55 | 10 | 2.8 | 1600 | 240 | 280 | 27 | 190 | 170 | 330 | 15 | 4.8 | 0.94 | 1200 |
| 7 | 2.6 | 0.53 | 16 | 13 | 1.1 | 0.61 | 0.43 | 0.88 | 2.1 | 0.93 | 0.45 | 7.6 | 0.44 | 0.81 | 3.0 | 21 | 5.6 | 0.68 | 1.0 |
| 8 | 0.19 | 0.06 | <5 | 3.0 | 0.13 | 0.13 | <0.1 | <0.2 | <0.2 | <0.2 | <0.1 | <2 | 0.10 | <0.1 | 0.16 | 2.2 | 1.3 | <0.2 | <0.2 |
| 10 | 110 | 140 | 44 | 58 | 120 | 5.7 | 1.4 | 6.5 | 230 | 2.6 | 2.7 | 22 | 1.7 | 1.0 | 5.9 | 50 | 19 | 59 | 14 |
| 11 | 1.9 | 2.1 | 8.2 | 44 | 1.6 | 0.52 | 0.02 | 3.2 | 27 | 1.2 | 0.80 | 16 | 0.42 | 0.40 | 4.0 | 20 | 1.1 | 0.58 | 1.2 |
| 12 | | | | | | | | | | | | | | | | | | | |
| 13 | 4.1 | 3.3 | 8.8 | 26 | 2.8 | 1.1 | 0.19 | 2.4 | 120 | 1.1 | 0.71 | <2 | 0.63 | 0.40 | 2.0 | 3.5 | <1 | 1.5 | 3.0 |
| 14 | | | | | | | | | | | | | | | | | | | |
| 15 | 280 | 240 | 96 | 51 | 250 | 160 | 3.7 | 120 | 570 | 100 | 11 | 19 | 6.7 | 5.1 | 120 | 83 | 19 | 1.2 | 2.6 |
| 16 | | | | | | | | | | | | | | | | | | | |
| 17 | 8.2 | 320 | 1600 | 1400 | 4.4 | 1.7 | 1.9 | 8.8 | 750 | 4.1 | 2.7 | 1200 | 2.3 | 1.4 | 190 | 1500 | 1100 | 1.2 | 0.35 |
| 18 | 19 | 75 | 25 | 54 | 66 | 4.8 | 1.0 | 15 | 410 | 6.6 | 5.5 | 5.9 | 3.8 | 37 | 100 | 16 | 19 | 2.5 | 3.1 |
| 19 | 8.8 | 4.9 | 9.9 | 9.3 | 4.1 | 1.9 | 0.27 | 4.3 | 97 | 1.8 | 1.6 | 2.4 | 1.3 | 0.73 | 2.6 | 7.6 | 7.8 | 1.9 | 1.6 |
| 20 | 0.25 | 0.15 | <5 | 13 | 0.15 | 0.11 | <0.1 | <0.2 | 0.52 | 0.30 | <0.1 | <2 | <0.1 | <0.1 | <0.1 | <2 | 1.8 | <0.2 | <0.2 |
| 21 | 0.12 | <0.05 | <5 | <2 | <0.1 | <0.1 | <0.1 | <0.2 | <0.2 | <0.2 | 0.04 | <2 | <0.1 | <0.1 | <0.1 | <2 | <1 | <0.2 | <0.2 |
| 22 | 0.51 | 0.30 | <5 | 24 | 0.27 | 0.16 | <0.1 | 0.21 | 0.83 | 0.26 | <0.1 | <2 | <0.1 | <0.1 | 0.14 | <2 | <1 | 0.21 | 0.28 |
| 23 | 0.73 | 0.50 | <5 | <2 | 0.45 | 0.27 | 0.19 | 0.22 | 0.63 | <0.2 | <0.1 | <2 | <0.1 | <0.1 | 0.13 | <2 | <1 | 0.24 | 0.47 |
| 24 | 1.3 | 0.82 | <5 | <2 | 0.69 | 0.31 | <0.1 | 0.22 | 4.4 | 0.48 | 0.25 | 2.8 | <0.1 | <0.1 | 0.26 | <2 | 230 | 0.49 | 0.85 |
| 25 | 1.9 | 1.6 | 6.1 | 2.8 | 1.5 | 0.54 | <0.1 | 0.85 | 20 | <0.2 | 0.5 | <2 | 0.35 | 0.21 | 1.0 | 13 | 48 | 1.6 | 2.0 |
| 26 | 520 | 490 | 350 | 290 | 570 | 300 | 87 | 340 | 1900 | 210 | 270 | 30 | 210 | 150 | 320 | 190 | 150 | 250 | 390 |
| 27 | 110 | 100 | 38 | 56 | 150 | 69 | 0.92 | 5.2 | 250 | 3.2 | 45 | 2.4 | 2.0 | 0.73 | 100 | 49 | 75 | 3.1 | 3.3 |
| 28 | 0.66 | 0.52 | <5 | <2 | 0.38 | 0.16 | <0.1 | 0.5 | 4.3 | 0.37 | 0.19 | <2 | 0.19 | <0.1 | 0.29 | <2 | 12 | 0.51 | 2.1 |
| 29 | 720 | 570 | 190 | 290 | 640 | 370 | 88 | 600 | 3000 | 430 | 450 | 37 | 330 | 280 | 550 | 130 | 3.4 | 2000 | 560 |
| 30 | 14 | 58 | 22 | 9.9 | 65 | 3.5 | 0.65 | 3.5 | 94 | 1.4 | 1.2 | 3.4 | 1.0 | 0.53 | 3.2 | 14 | 1.6 | 1.2 | 2.0 |
| 31 | <0.1 | <0.05 | 14 | 12 | <0.1 | <0.1 | <0.1 | <0.2 | <0.2 | <0.2 | 0.1 | 6.0 | <0.1 | <0.1 | <0.1 | 13 | 250 | 0.9 | 1.4 |

^{a}In mmol ester hydrolyzed/min/mg protein. Bold numbers highlight all specific activities > 100 mmol ester hydrolyzed/min/mg protein.

Good substrates showed a specific activity >100 mmol ester hydrolyzed/mg protein/min, while very good substrates showed a specific activity >1000. The best substrates were activated esters, which are also chemically the most reactive: vinyl esters (1-6), ethyl trifluoroacetate (17), phenyl acetate (26). Among the vinyl and ethyl esters with different chain lengths, most of the _{ThermoGen} esterases appear to favor intermediate chain lengths (butanoate and hexanoate), while E018b appears to strongly favor acetate esters. The sterically hindered vinyl pivaloate (11) was a poor substrate for all the esterases, while vinyl benzoate (10) was a good substrate for several esterases. Polar esters (e.g., 20, 21, 31) were usually poor substrates. Ethyl butyrate (13) reacted much slower that the more hydrophobic tributyrin (29). This preference for hydrophobic substrates suggests that these esterases may be related to lipases.

5. Acyl Chain Length Selectivity

The true selectivity of the _{ThermoGen} esterases toward different acyl chain length was determined using a competitive experiment. Resorufin esters were used as the competitive substrate as explained above and normalized the results to hexanoate =100, Table 7. The true selectivities were similar to the estimated selectivities.

TABLE 7
Acyl chain length
selectivity of ThermoGen esterases toward vinyl and ethyl esters (hexanoate = 100).^{a}

| | vinylesters | | | | | ethyl esters | | | |
|---|---|---|---|---|---|---|---|---|---|
| esterase | C2,1 | C3,2 | C4,3 | C6,4 | C8,5 | C10,6 | C2,12 | C6,14 | C10,16 |
| E001 | 0.11^{c} | 0.81 | 3 | 100 | 12 | 2.8^{c} | nd | nd | nd |
| E002 | 0.48^{d} | 2.2 | 7.2 | 100 | 20 | 9.1^{c} | <0.022^{b} | 100^{d} | 230^{d} |
| E003 | 1.4^{d} | 7.3 | 19 | 100 | 46 | 22^{c} | <0.056 | 100^{d} | 55^{d} |
| E004 | 0.19^{d} | 1.4 | 16 | 100 | 16 | 2.2^{d} | nd | nd | nd |
| E005 | 0.80^{d} | 1.9 | 5 | 100 | 17 | 8.4^{c} | nd | nd | nd |
| E006 | 0.0043^{c} | 0.86 | 2.7 | 100 | 11 | 2.3^{c} | <0.043^{b} | 100^{d} | 23^{d} |
| E007 | 0.52^{d} | 1.1 | 0.43 | 100 | 17 | 3.3^{c} | nd | nd | nd |
| E008 | 0.0096^{d} | 0.056 | 3.5 | 100 | 4.7 | 0.13^{d} | nd | nd | nd |
| E009 | 0.052^{d} | 0.37 | 3.5 | 100 | 4.4 | 0.68^{d} | nd | nd | nd |
| E010 | 0.068^{d} | 0.88^{d} | 36 | 100 | 5.2 | 1.5^{d} | nd | nd | nd |
| E011 | 0.027^{d} | 0.19 | 33 | 100 | 4.1 | 0.43^{d} | <0.088^{b} | 100^{d} | 15^{d} |
| E012 | 0.070^{c} | 1.2 | 49 | 100 | 16 | 2.5^{c} | nd | nd | nd |
| E013 | 0.0042^{d} | 0.030 | 4.0 | 100 | 5.4 | 0.056^{d} | nd | nd | nd |
| E014 | 0.0084^{d} | 1.7^{d} | 3.4 | 100 | 4.7 | 3.8^{d} | nd | nd | nd |
| E015 | 0.072^{d} | 1.1^{d} | 3.7 | 100 | 5.2 | 1.9^{d} | nd | nd | nd |
| E016 | 0.34^{d} | 53 | 16 | 100 | 4.1 | 6.5^{c} | nd | nd | nd |
| E018^{b} | 7600^{c} | 680^{c} | 74^{c} | 100^{c} | 217^{c} | 112^{c} | 2100^{c,e} | 100^{d} | 640^{c} |
| E019 | 0.12^{c} | 0.98 | 39 | 100 | 41 | 6.5^{c} | nd | nd | nd |
| E020 | 0.040^{d} | 0.92 | 3.2 | 100 | 17 | 2.6^{c} | nd | nd | nd |
| AcEf | 850^{c} | 370^{c} | 144^{c} | 100^{c} | 150^{c} | 160^{c} | nd | nd | nd |

^{a}True selectivities (estimated error limits are ±10-30%) measured using resorufin acetate as the reference compound unless otherwise noted.
nd = not determined.
^{b}Substrate reacted much slower than all available resorufin esters. The value listed is an estimated upper limit.
^{c}resorufin isobutyrate as the reference compound.
^{d}resorufin pivaloate as the reference compound
^{e}estimated error limits are larger, ±50%.
^{f}Acetyl esterase from orange peel.

Among the vinyl esters, the hexanoate was the best substrate for all except E018b, where the acetate was the best substrate. E018b favored vinyl acetate 76-fold over vinyl hexanoate. The acetate was a very poor substrate for many esterases (e.g., E006, E008, E0013, E0014). For example, E006 favored vinyl hexanoate >1000-fold over vinyl acetate. Among more limited data for the ethyl esters, the hexanoate vs. acetate selectivity was similar: E018b favored ethyl acetate 21-fold over ethyl hexanoate, while E006 favored ethyl hexanoate >1000-fold over ethyl acetate. Confirming the selectivity of E018b for acetyl groups, E018b catalyzed hydrolysis of the acetate esters, but not butyrate or other esters, in the enantiomer pair library, see below. As suggested by the initial screening, true selectivity measurements showed that both vinyl pivaloate and vinyl benzoate were poor substrates.

There were some small differences in chain length selectivity for the vinyl vs. ethyl esters. For example, E002 favored vinyl hexanoate 11-fold over vinyl decanoate, but the same enzyme favored ethyl decanoate 2.3-fold over ethyl hexanoate.

For comparison, chain length selectivity was also measured for the acetyl esterase from orange peel (AcE), which is commercially available from Sigma it has been used several times in organic synthesis for removal of acetyl groups (Waldmann, et al. Bioorg. Med. Chem. 1994, 2, 477-482; Waldmann, H.; Heuser, A.; Reidel, A. Synlett 1994, 65-67). For the vinyl esters AcE favors an acetyl only 7.7-fold over a hexanoyl group, approximately a ten-fold lower selectivity than E018b.

6. Estimated Enantioselectivity

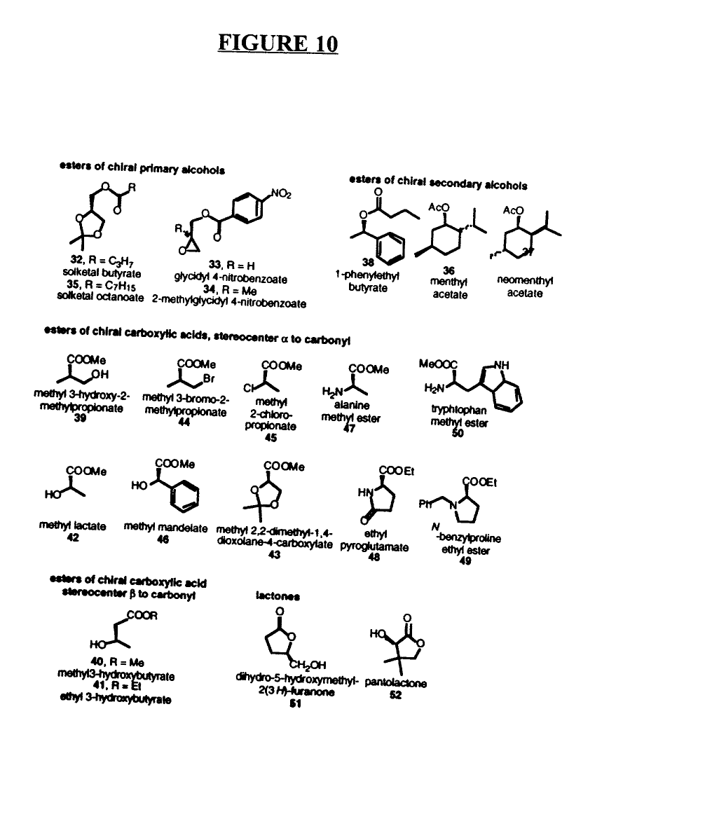

To evaluate the potential of the _{ThermoGen} esterases for enantioselective reactions, the initial rates of hydrolysis of the esterases were measured using twenty-one pairs of enantiomers, Scheme 2, FIG. 10 and Tables 8-12. The ratio of these initial rates is an estimated enantioselectivity.

TABLE 8
Specific activities and
estimated enantioselectivitities of ThermoGen esterases toward esters of chiral primary alcohols.^{a}

| | (R)- | (S)- | Est. | (R)- | (S)- | Est. | (R)- | (S)- | Est. | (R)- | (S)- | Est |
|---|---|---|---|---|---|---|---|---|---|---|---|---|
| | 32R | 32S | E^{b} | 35 | 35 | E^{b} | 33 | 33 | E^{b} | 34 | 34 | E^{b} |
| E001 | 9.4 | 7.1 | 1.3 | 200 | 140 | 1.4 | 12 | 12.5 | 1.0 | 16 | 40 | 2.6 |
| E002 | 7.3 | 5.6 | 1.3 | 250 | 160 | 1.6 | 7.8 | 8.5 | 1.1 | 36 | 85 | 2.4 |
| E003 | 19 | 15 | 1.3 | 140 | 92 | 1.5 | 18 | 13 | 1.4 | 16 | 27 | 1.7 |
| E004 | 27 | 20 | 1.4 | 180 | 160 | 1.1 | 34 | 34 | 1.0 | 68 | 53 | 1.3 |
| E005 | 5.8 | 4.4 | 1.3 | 300 | 170 | 1.8 | 6.9 | 7.2 | 1.0 | 44 | 96 | 2.2 |
| E006 | 2.4 | 1.8 | 1.3 | 100 | 61 | 1.7 | 2.5 | 2.9 | 1.2 | 3.0 | 7.9 | 2.7 |
| E007 | 0.44 | 0.32 | 1.4 | 4.0 | 2.5 | 1.6 | 0.44 | 0.46 | 1.0 | 0.43 | 1.3 | 2.9 |
| E008 | 6.0 | 4.2 | 1.4 | 93 | 150 | 1.6 | 6.4 | 8.9 | 1.4 | 17 | 16 | 1.0 |
| E009 | 150 | 110 | 1.4 | 300 | 490 | 1.6 | 150 | 190 | 1.2 | 360 | 320 | 1.2 |
| E010 | 2.8 | 2.7 | 1.0 | 81 | 110 | 1.4 | 2.9 | 3.6 | 1.3 | 6.4 | 6.0 | 1.1 |
| E011 | 2.2 | 2.2 | 1.0 | 58 | 94 | 1.6 | 2.6 | 3.0 | 1.2 | 6.1 | 5.3 | 1.1 |
| E012 | 3.2 | 3.0 | 1.1 | 19 | 18 | 1.0 | 5.1 | 4.3 | 1.2 | 14 | 9.6 | 1.5 |
| E013 | 1.5 | 1.1 | 1.3 | 94 | 84 | 1.1 | 1.4 | 2.0 | 1.4 | 3.0 | 3.0 | 1.0 |
| E014 | 1.4 | 1.2 | 1.2 | 83 | 76 | 1.1 | 1.2 | 1.4 | 1.2 | 3.3 | 3.4 | 1.0 |
| E015 | 7.2 | 6.3 | 1.2 | 230 | 190 | 1.2 | 7.6 | 9.2 | 1.2 | 18 | 18 | 1.0 |
| E016 | 14 | 9.3 | 1.5 | 93 | 57 | 1.6 | 13 | 10 | 1.3 | 11 | 26 | 2.3 |
| E018^{b} | <1.5 | <1.5 | ^{c} | <1.5 | <1.5 | ^{c} | <1.5 | <1.5 | ^{c} | <1.5 | <1.5 | ^{c} |
| E019 | 3.1 | 2.9 | 1.1 | 120 | 76 | 1.6 | 3.8 | 3.1 | 1.3 | 2.7 | 4.8 | 1.8 |
| E020 | 4.5 | 4.5 | 1.0 | 120 | 85 | 1.5 | 6.1 | 4.8 | 1.3 | 3.9 | 8.9 | 2.3 |

^{a}Specific activity in mmol ester hydrolyzed/mg protein/min. Rates_100 are bolded for emphasis.
^{b}Estimated enantioselectivity is the ratio of the initial rates of hydrolysis of the two enantiomers (rate for fast enantiomer/rate for slow enantiomer).
^{c}Could not be measured because the substrate did not react.

TABLE 9
Specific activities and estimated
enantioselectivitities of ThermoGen esterases toward
esters of chiral secondary alcohols.^{a}

| | (R)- | (S)- | Est. | (R)- | (S)- | Est. | (R)- | (S)- | Est. |
|---|---|---|---|---|---|---|---|---|---|
| | 38 | 38 | E^{b} | 36 | 36 | E^{b} | 37 | 37 | E^{b} |
| E001 | 24 | 2.0 | 11 | 0.82 | 1.8 | 2.2 | 0.52 | 0.40 | 1.3 |
| E002 | 110 | 21 | 5.1 | 0.54 | 1.4 | 2.6 | 0.08 | 0.08 | 1.1 |
| E003 | 37 | 5.6 | 6.6 | 4.2 | 7.0 | 1.6 | 3.5 | 3.2 | 1.1 |
| E004 | 160 | 28 | 5.7 | 62 | 120 | 2.0 | 3.8 | 4.9 | 1.3 |
| E005 | 4.0 | 0.31 | 13 | 0.53 | 1.3 | 2.4 | 0.21 | 0.19 | 1.1 |
| E006 | 5.0 | 0.27 | 18 | 0.45 | 0.54 | 1.2 | 0.27 | 0.25 | 1.1 |
| E007 | 1.2 | 0.23 | 5.2 | 0.22 | 0.26 | 1.2 | 0.14 | 0.14 | 1.0 |
| E008 | 260 | 57 | 4.6 | 150 | 200 | 1.0 | 0.32 | 0.41 | 1.3 |
| E009 | 900 | 120 | 6.7 | 930 | 930 | 1.0 | 0.65 | 1.3 | 2.0 |
| E010 | 16 | 1.4 | 11 | 150 | 130 | 1.1 | 0.28 | 0.32 | 1.1 |
| E011 | 130 | 15 | 8.6 | 180 | 150 | 1.2 | 0.14 | 0.19 | 1.4 |
| E012 | 12 | <2 | >9.3 | 16 | 19 | 1.2 | 8.1 | 7.8 | 1.1 |
| E013 | 5.3 | 0.46 | 12 | 76 | 70 | 1.5 | 0.20 | 0.23 | 1.2 |
| E014 | 5.0 | 0.32 | 15 | 34 | 47 | 1.4 | 0.13 | 0.14 | 1.1 |
| E015 | 24 | 1.5 | 16 | 93 | 150 | 1.7 | 0.25 | 0.38 | 1.5 |
| E016 | 23 | 3.5 | 6.4 | 1.5 | 1.4 | 1.1 | 5.2 | 5.3 | 1.0 |
| E018^{b} | 1.3 | 1.3 | 1.0 | 11 | 6.2 | 1.7 | 6.9 | 6.1 | 1.1 |
| E019 | 7.5 | 0.45 | 17 | 0.44 | 0.68 | 1.6 | 0.20 | 0.19 | 1.0 |
| E020 | 9.2 | 0.63 | 15 | 0.64 | 1.1 | 1.7 | 0.31 | 0.34 | 1.1 |

^{a}Specific activity in mmol ester hydrolyzed/mg protein/mm. Rates_100 are bolded for emphasis.
^{b}Estimated enantioselectivity is the ratio of the initial rates of hydrolysis of the two enantiomers (rate for fast enantiomer/rate for slow enantiomer).
^{c}Could not be measured because the substrate did not react.

TABLE 10
Specific activities and estimated enantioselectivitities
of ThermoGen esterases toward esters of chiral carboxylic acids
(stereocenter at the a-position).^{a}

| | (R)- | (S)- | Est. | (R)- | (S)- | Est. | (R)- | (S)- | Est. | (R)- | | Est. | | | Est. | | | Est. |
|---|---|---|---|---|---|---|---|---|---|---|---|---|---|---|---|---|---|---|
| | 39 | 39 | E^{b} | 44 | 44 | E^{b} | 45 | 45 | E^{b} | 47 | (S)-47 | E^{b} | (R)-50 | (S)-50 | E^{b} | (R)-42 | (S)-42 | E^{b} |
| E001 | <0.1 | <0.1 | ^{c} | 0.38 | 0.18 | 2.1 | 2.3 | 20 | 8.5 | <0.1 | <0.1 | ^{c} | <0.1 | 0.22 | >2.2 | 0.60 | 0.51 | 1.2 |
| E002 | <0.05 | <0.05 | ^{c} | 0.19 | 0.053 | 2.1 | 50 | 100 | 3.4 | <0.05 | <0.05 | ^{c} | 0.02 | 0.17 | 8.5 | 0.45 | 0.30 | 1.6 |
| E003 | 6.7 | 5.4 | 1.2 | <2.5 | <2.5 | ^{c} | 11 | 38 | 3.4 | <2.5 | <2.5 | ^{c} | 4.3 | 7.2 | 1.7 | 15 | 15 | 1.0 |
| E004 | 27 | 29 | 1.1 | 3.0 | <2 | >2.3 | 13 | 31 | 2.4 | <2 | <2 | ^{c} | <2 | <2 | ^{c} | 46 | 39 | |
| E005 | <0.1 | <0.1 | ^{c} | 0.035 | 0.015 | 2.1 | 56 | 120 | 2.2 | <0.1 | <0.1 | ^{c} | <0.1 | 0.73 | >7.3 | 0.37 | 0.16 | 2.4 |
| E006 | <0.1 | <0.1 | ^{c} | <0.1 | <0.1 | ^{c} | 0.81 | 4.9 | 6.1 | <0.1 | <0.1 | ^{c} | <0.1 | 0.28 | >2.8 | 0.20 | 0.12 | 1.6 |
| E007 | <0.06 | <0.06 | ^{c} | <0.06 | <0.06 | ^{c} | 0.21 | 1.0 | 5.1 | <0.06 | <0.06 | ^{c} | <0.06 | <0.06 | ^{c} | <0.06 | <0.06 | ^{c} |
| E008 | <0.2 | <0.2 | ^{c} | 0.63 | 0.21 | 3.0 | 3.1 | 7.3 | 2.3 | 0.28 | 0.66 | 2.4 | <0.2 | <0.2 | ^{c} | 0.42 | 0.46 | 1.1 |
| E009 | <0.2 | <0.2 | ^{c} | 3.1 | 0.79 | 4.0 | 56 | 180 | 3.2 | <0.2 | <0.2 | ^{c} | <0.2 | 0.33 | >1.6 | 1.0 | 1.9 | 1.8 |
| E010 | <0.1 | <0.1 | ^{c} | <0.1 | <0.1 | ^{c} | 1.1 | 2.5 | 2.3 | <0.1 | <0.1 | ^{c} | <0.1 | <0.1 | ^{c} | 0.19 | 0.21 | 1.1 |
| E011 | <0.05 | <0.05 | ^{c} | <0.05 | <0.05 | ^{c} | 28 | 45 | 1.6 | <0.05 | <0.05 | ^{c} | <0.05 | <0.05 | ^{c} | 0.050 | 0.080 | 1.5 |
| E012 | 29 | 28 | 1.1 | <2 | <2 | ^{c} | <2 | <2 | ^{c} | <2 | <2 | ^{c} | <2 | <2 | ^{c} | 34 | 37 | 1.1 |
| E013 | <0.05 | <0.05 | ^{c} | <0.05 | <0.05 | ^{c} | 0.58 | 1.5 | 2.6 | <0.05 | <0.05 | ^{c} | <0.05 | <0.05 | ^{c} | <0.05 | <0.05 | ^{c} |
| E014 | <0.05 | <0.05 | ^{c} | 0.023 | 0.015 | 1.5 | 0.35 | 0.97 | 2.8 | <0.05 | <0.05 | ^{c} | <0.05 | <0.05 | ^{c} | <0.05 | <0.05 | ^{c} |
| E015 | <0.05 | <0.05 | ^{c} | 0.24 | 0.06 | 4.0 | 2.9 | 7.5 | 2.5 | <0.05 | <0.05 | ^{c} | <0.05 | <0.05 | ^{c} | 0.095 | 0.25 | 2.6 |
| E016 | 27 | 29 | 1.1 | 1.2 | 1.2 | 1.0 | 9.6 | 28 | 2.9 | <0.5 | <0.5 | ^{c} | <0.5 | 1.7 | >3.4 | 37 | 34 | 1.1 |
| E018^{b} | <1.5 | <1.5 | ^{c} | <1.5 | <1.5 | ^{c} | <1.5 | <1.5 | ^{c} | <1.5 | <1.5 | ^{c} | <1.5 | <1.5 | ^{c} | <1.5 | <1.5 | ^{c} |
| E019 | 0.10 | 0.16 | 1.6 | <0.1 | <0.1 | ^{c} | 0.74 | 5.4 | 7.3 | <0.1 | <0.1 | ^{c} | <0.1 | <0.1 | ^{c} | 0.25 | 0.16 | 1.6 |
| E020 | 0.64 | 0.68 | 1.1 | <0.2 | <0.2 | ^{c} | 1.7 | 7.5 | 4.5 | <0.2 | <0.2 | ^{c} | <0.2 | <0.2 | ^{c} | 0.41 | 0.29 | 1.6 |

^{a}Specific activity in mmol ester hydrolyzed/mg protein/min. Rates_100 are bolded for emphasis.
^{b}Estimated enantioselectivity is the ratio of the initial rates of hydrolysis of the two enantiomers (rate for fast enantiomer/rate for slow enantiomer).
^{c}Could not be measured becuase the substrate did not react.

TABLE 11
Specific activities and estimated enantioselectivitities of ThermoGen esterases toward esters of chiral carboxylic
acids (stereocenter at the a-position).a

| | | | Est. | | | Est. | | | Est. | | | Est. |
|---|---|---|---|---|---|---|---|---|---|---|---|---|
| | (R)-46 | (S)-46 | Eb | (R)-43 | (S)-43 | Eb | (R)-48 | (S)-48 | Eb | (R)-49 | (S)-49 | Eb |
| E001 | 0.13 | 0.14 | 1.1 | 3.2 | 0.4 | 8.1 | 0.13 | 0.14 | 1.0 | 0.34 | 0.42 | 1.2 |
| E002 | <0.05 | <0.05 | c | 50 | 43 | 1.2 | <0.05 | <0.05 | c | <0.05 | <0.05 | c |
| E003 | <2.5 | <2.5 | c | 8.2 | 5.6 | 1.5 | <2.5 | <2.5 | c | <2.5 | <2.5 | c |
| E004 | <2 | <2 | c | <2 | <2 | 1.2 | <2 | <2 | c | <2 | <2 | c |
| E005 | <0.1 | <0.1 | c | 61 | 52 | 1.2 | <0.1 | <0.1 | c | <0.1 | <0.1 | c |
| E006 | <0.1 | <0.1 | c | 0.75 | 0.11 | 6.6 | <0.1 | <0.1 | c | 0.15 | 0.14 | 1.1 |
| E007 | <0.06 | <0.06 | c | <0.06 | <0.06 | c | <0.06 | <0.06 | c | 0.10 | 0.17 | 1.7 |
| E008 | <0.2 | <0.2 | c | 0.26 | 0.22 | 1.2 | 0.70 | 1.1 | 1.5 | 0.81 | 0.90 | 1.1 |
| E009 | 0.33 | 0.26 | 1.3 | 0.63 | 0.37 | 1.7 | 0.80 | 0.85 | 1.1 | 0.69 | 0.91 | 1.3 |
| E010 | <0.1 | <0.1 | c | <0.1 | <0.1 | c | 0.17 | 0.25 | 1.4 | 0.17 | 0.25 | 1.4 |
| E011 | <0.05 | <0.05 | c | <0.05 | <0.05 | c | <0.05 | <0.05 | c | 0.12 | 0.10 | 1.3 |
| E012 | <2 | <2 | c | <2 | <2 | c | <2 | <2 | c | <2 | <2 | c |
| E013 | <0.05 | <0.05 | c | <0.05 | <0.05 | c | <0.05 | <0.05 | c | 0.11 | 0.13 | 1.3 |
| E014 | <0.05 | <0.05 | c | <0.05 | <0.05 | c | <0.05 | <0.05 | c | <0.05 | <0.05 | c |
| E015 | <0.05 | <0.05 | c | 0.13 | 0.060 | 2.1 | <0.05 | <0.05 | c | 0.20 | 0.18 | 1.1 |
| E016 | 1.2 | 1.5 | 1.2 | 5.3 | 1.3 | 4.1 | 0.90 | 0.75 | 1.2 | 6.7 | 8.4 | 1.2 |
| E018b | <1.5 | <1.5 | c | <1.5 | <1.5 | c | <1.5 | <1.5 | c | <1.5 | <1.5 | c |
| E019 | <0.1 | <0.1 | c | 0.90 | 0.10 | 8.9 | <0.1 | <0.1 | c | 0.23 | 0.21 | 1.1 |
| E020 | <0.2 | <0.2 | c | 1.3 | 0.25 | 5.4 | <0.2 | <0.2 | c | <0.2 | <0.2 | c |

a Specific activity in mmol ester hydrolyzed/mg protein/min. Rates_100 are bolded for emphasis.
b Estimated enantioselectivity is the the ratio of the initial rates of hydrolysis of the two enantiomers (rate for fast enantiomer/rate for slow enantiomer).
c Could not be measured because the substrate did not react.

TABLE 12
Specific activities and estimated enantioselectivitities of ThermoGen esterases toward esters of chiral carboxylic
acids (sterocenter at the b-position) and lactones.a

| | | | Est. | | | Est. | | | Est. | | | Est. |
|---|---|---|---|---|---|---|---|---|---|---|---|---|
| | (R)-40 | (S)-40 | Eb | (R)-41 | (S)-41 | Eb | (R)-51 | (S)-51 | Eb | (R)-52 | (S)-52 | Eb |
| E001 | <0.1 | <0.1 | c | 0.16 | 0.15 | 1.1 | <0.1 | <0.1 | c | <0.1 | <0.1 | c |
| E002 | <0.05 | <0.05 | c | <0.05 | <0.05 | c | <0.05 | <0.05 | c | <0.05 | <0.05 | c |
| E003 | <0.25 | <0.25 | c | 11 | 9.4 | 1.2 | 10 | 11 | 1.1 | 7.7 | 11 | 1.4 |
| E004 | 41 | 39 | 1.1 | 24 | 33 | 1.4 | <2 | <2 | c | <2 | <2 | c |
| E005 | <0.1 | <0.1 | c | 0.15 | 0.22 | 1.4 | <0.1 | <0.1 | c | <0.1 | <0.1 | c |
| E006 | <0.1 | <0.1 | c | <0.1 | <0.1 | c | <0.1 | <0.1 | c | <0.1 | <0.1 | c |
| E007 | <0.06 | <0.06 | c | <0.06 | <0.06 | c | <0.06 | <0.06 | c | <0.06 | <0.06 | c |
| E008 | <0.2 | <0.2 | c | 0.27 | 0.25 | 1.1 | <0.2 | <0.2 | c | <0.2 | <0.2 | c |
| E009 | 0.30 | 0.35 | 1.2 | 0.34 | 0.47 | 1.4 | <0.2 | <0.2 | c | <0.2 | <0.2 | c |
| E010 | 0.24 | 0.24 | 1.0 | <0.1 | <0.1 | c | <0.1 | <0.1 | c | <0.1 | <0.1 | c |
| E011 | <0.05 | <0.05 | c | <0.05 | <0.05 | c | <0.05 | <0.05 | c | <0.05 | <0.05 | c |
| E012 | 29 | 31 | 1.1 | 21 | 28 | 1.3 | <2 | <2 | c | <2 | <2 | c |
| E013 | <0.05 | <0.05 | c | <0.05 | <0.05 | c | <0.05 | <0.05 | c | <0.05 | <0.05 | c |
| E014 | <0.05 | <0.05 | c | <0.05 | <0.05 | c | <0.05 | <0.05 | c | <0.05 | <0.05 | c |
| E015 | <0.05 | <0.05 | c | <0.05 | <0.05 | c | <0.05 | <0.05 | c | <0.05 | <0.05 | c |
| E016 | 27 | 28 | 1.0 | 24 | 32 | 1.3 | 1.0 | 1.1 | 1.0 | 1.0 | 1.0 | 1.0 |
| E018b | <1.5 | <1.5 | c | <1.5 | <1.5 | c | <1.5 | <1.5 | c | <1.5 | <1.5 | c |
| E019 | <0.1 | <0.1 | c | <0.1 | <0.1 | c | <0.1 | <0.1 | c | <0.1 | <0.1 | c |
| E020 | <0.2 | <0.2 | c | <0.2 | <0.2 | c | <0.2 | <0.2 | c | <0.2 | <0.2 | c |

a Specific activity in mmol ester hydrolyzed/mg protein/min. Rates_100 are bolded for emphasis.
b Estimated enantioselectivity is the ratio of the initial rates of hydrolysis of the two enantiomers (rate for fast enantiomers/rate for slow enantiomer).
c Could not be measured because the substrate did not react.

Substrates with with the chirality in the alcohol portion (Tables 8 and 9) usually reacted faster than substrates than the chirality in the carboxylic acid portion (Tables 10-12). The poorest substrates—39, 47, 46, 48, 40, 51, 52—were polar molecules with the stereocenter in the carboxylic acid portion. The best substrates—32, 33, 35, 37—were nonpolar molecules with the stereocenter in the alcohol portion. This observation is consistent with the screening above which also showed that polar esters were poor substrates.

The estimated enantioselectivities were below two in most cases, but two substrates (38, 45) showed higher estimated enantioselectivities with most _{ThermoGen} esterases. In addition, six other substrates (34, 36, 43, 44, 47, 50) showed estimated enantioselectivities above two with several esterases.

7. True Enantioselectivity

The true enantioselectivity for five of these esters was measured using a resorufin ester as a reference compound as described above. Measurements of the enantioselectivity for substrates 44, 47, and 50 were not obtained because they reacted too slowly compared to the resorufin esters used as the reference compound. In other cases, when the slow enantiomer reacted much slower than the reference compound, we could only set a lower limit on the enantioselectivity. Results are summarized in Table 13, detailed data for selected measurements is in Table 8 above.

TABLE 13
Enantioselectivity of Thermogen esterases toward selected substratesa

| | 34 | 38 | 36 | 45 | 43 |
|---|---|---|---|---|---|
| E001 | 3.5 | >47 (R) | >2.4 (S) | 11 (S) | >3.4 (R) |
| | (S) | | | | |
| E002 | 3.9 | >120 (R) | >6.4 (S) | 7.8 (S) | nd |
| | (S) | | | | |
| E003 | nd | 14 (R) | nd | 6.8 (S) | nd |
| E004 | nd | 150 (R) | >68 (S) | 18 (S) | nr |
| E005 | 4.2 | >65 (R) | 2.0 (S) | 8.7 (S) | nd |
| | (S) | | | | |
| E006 | 4.0 | >33 (R) | nd | 12 (S) | >1.1 (R) |
| | (S) | | | | |
| E007 | 2.5 | 13 (R) | nd | 4.4 (S) | nr |
| | (S) | | | | |
| E008 | nd | >110 (R) | nd | >28 (S) | nd |
| E009 | nd | 180 (R) | nd | 8.4 (S) | nd |
| E010 | nd | >69 (R) | nd | >11 (S) | nr |
| E011 | nd | >140 (R) | nd | >30 (S) | nr |
| E012 | nd | 3.1 (R) | nd | nr | nr |
| E013 | nd | >120 (R) | nd | >14 (S) | nr |
| E014 | nd | >59 (R) | nd | 24 (S) | nr |
| E015 | nd | >320 (R) | nd | 17 (S) | nd |
| E016 | 3.5 | 13 (R) | nd | 5.8 (S) | >9.9 (R) |
| | (S) | | | | |
| E018b | nr | nd | nd | nr | nr |
| E019 | nd | >52 (R) | nd | 15 (S) | >1.3 (R) |
| E020 | 3.2 | >53 (R) | nd | 12 (S) | >1.7 (R) |
| | (S) | | | | |

anr = not determined either because the substrate did not react.
nd = not determined because the estimated enantioselectivity was <2. Unless otherwise noted, the true enantioselectivities were measured using resorufin aceate as a reference compound and have an estimated error of ±10-30%. In cases where the rate of the slow enantiomer was too slow to measure accurately, a lower limit for the enantioselectivity is given.

None of the esterases showed enantioselectivities above five for 34, an ester of a primary alcohol. Most of the esterases showed high enantioselectivity toward 1-phenylethyl butyrate, 38, an ester of a secondary alcohol. Esterases E002, E004, E005, E008, E009, E100, E011, E013, E014, E015, E019 and E020 all showed enantioselectivities >50. In addition, E001 and E006 may also be highly enantioselective, but our measured lower limits for the enantioselectivity were 47 and 33. Like other lipases and esterases, the _{ThermoGen} esterases favored the (R)-enantiomer of 38. One esterase, E004, showed high enantioselectivity (E>68) toward another secondary alcohol, menthyl acetate. In addition, two other esterases, E001 and E002, may also be highly enantioselective, but our measured lower limits for the enantioselectivity were low: 2.4 and 6.4, respectively. The favored enantiomer was (S), check and comment.

Most of the esterases showed moderate or better enantioselectivty toward methyl 2-chloropropanoate, 45. Esterases E008, E011, and E014 showed enantioselectivities above twenty-four. All esterases favored the (S)-enantiomer. Five esterases showed some enantioselectivity toward the dioxolane derivative 43. Because reactions were slow, the measured lower limits on enantioselectivity are low.

While a preferred form of the invention has been shown in the drawings and described, since variations in the preferred form will be apparent to those skilled in the art, the invention should not be construed as limited to the specific form shown and described, but instead is as set forth in the claims.

We claim:

1. A method for the quantitative screening of hydrolase for desired substrate activity using pH indicators which are sensitive to the release of protons from a chemical reaction in a reaction mixture, said method comprising selecting buffer and indicator conditions such that both have the same affinity for protons such that the relative amount of buffer protonated is proportional to the amount of indicator protonated as the pH of the reaction mixture shifts; combining in a reaction mixture said selected buffer, indicator, hydrolayse to be tested, and desired substrate to be tested; allowing the hydrolase to react with the substrate; and monitoring progress of the reaction by detection of change in the indicator.

2. A method of claim 1 wherein said buffer and said indicator have a pKₐ within about 0.1 of each other.

3. A method of claim 1 wherein said buffer is BES (N,N-bis[2-hydroxyethyl]-2-aminoethanesulfonic acid.

4. A method of claim 1 wherein said indicator is 4-nitrophenol.

5. A method of claim 1 wherein said substrate concentration is in the range of about 0.1 mM to 2.0 mM.

6. A method of claim 1 wherein an additional organic cosolvent is added.

7. A method of claim 6 wherein said cosolvent is acetonitrile or dimethyl sulfoxide.

8. A method of claim 7 wherein said cosolvent is present at up to 10% of volume.
